SEQUENCE LISTING (73 sequences)

SEQ ID NO: 1 - DNA, 20 bp, Artificial
  Primer
agagtttgatcctggctcag

SEQ ID NO: 2 - DNA, 27 bp, Artificial
  Primer
taccgtcaccataaggcttcgtcccta

SEQ ID NO: 3 - DNA, 18 bp, Artificial
  Primer
gtttcccaggcttatccc

SEQ ID NO: 4 - DNA, 52 bp, Artificial
  Primer
ggagcacgctatcccgttagaccagaggagcccatttggtagtgaggcaggt

SEQ ID NO: 5 - DNA, 45 bp, Artificial
  Primer
ggagcacgctatcccgttagactggacgccggtggtcgtgctcaa

SEQ ID NO: 6 - DNA, 43 bp, Artificial
  Primer
ggagcacgctatcccgttagaccacgcgcacgtgcccgtccca

SEQ ID NO: 7 - DNA, 45 bp, Artificial
  Primer
ggagcacgctatcccgttagaccgctggctggcaaggtcctacgc

SEQ ID NO: 8 - DNA, 41 bp, Artificial
  Primer
ggagcacgctatcccgttagacgccccggcccagccaccat

SEQ ID NO: 9 - DNA, 45 bp, Artificial
  Primer
cgctgccaactaccgcacatgggtcccacggaaatctgtctctgt

SEQ ID NO: 10 - DNA, 44 bp, Artificial
  Primer
cgctgccaactaccgcacatgcctctgccgccctccaggacctc

SEQ ID NO: 11 - DNA, 45 bp, Artificial
  Primer
cgctgccaactaccgcacatgctctcgctccgcacctcgcgcaga

SEQ ID NO: 12 - DNA, 48 bp, Artificial
  Primer
cgctgccaactaccgcacatgccctcggcccctgcactgtttcccaga

SEQ ID NO: 13 - DNA, 45 bp, Artificial
  Primer
cgctgccaactaccgcacatgtggctagggagcaggctggggact

SEQ ID NO: 14 - DNA, 22 bp, Artificial
  Primer
ggagcacgctatcccgttagac

SEQ ID NO: 15 - DNA, 21 bp, Artificial
  Primer
cgctgccaactaccgcacatg

SEQ ID NO: 16 - DNA, 20 bp, Artificial
  Primer
ggctgacctgttctctgccg

SEQ ID NO: 17 - DNA, 20 bp, Artificial
  Primer
ggaaccctgagagcagcttc

SEQ ID NO: 18 - DNA, 27 bp, Artificial
  Primer
aatgatgagaacctgtgcatagtggtg

SEQ ID NO: 19 - DNA, 28 bp, Artificial
  Primer
aatgatgagaacctgtcgcatagtggtg

SEQ ID NO: 20 - DNA, 28 bp, Artificial
  Primer
aatgatgagaacctggtgcatagtggtg

SEQ ID NO: 21 - DNA, 29 bp, Artificial
  Primer
aatgatgagaacctggtcgcatagtggtg

SEQ ID NO: 22 - DNA, 27 bp, Artificial
  Primer
aatgatgagaacctgggcatagtggtg

SEQ ID NO: 23 - DNA, 28 bp, Artificial
  Primer
aatgatgagaacctggcgcatagtggtg

SEQ ID NO: 24 - DNA, 26 bp, Artificial
  Primer
aatgatgagaacctggcatagtggtg

SEQ ID NO: 25 - DNA, 28 bp, Artificial
  Primer
aatgatgagaacctgctgcatagtggtg

SEQ ID NO: 26 - DNA, 29 bp, Artificial
  Primer
aatgatgagaacctgctcgcatagtggtg

SEQ ID NO: 27 - DNA, 27 bp, Artificial
  Primer
aatgatgagaacctgcgcatagtggtg

SEQ ID NO: 28 - DNA, 28 bp, Artificial
  Primer
aatgatgagaacctgccgcatagtggtg

SEQ ID NO: 29 - DNA, 28 bp, Artificial
  Primer
aatgatgagaacctgatgcatagtggtg

SEQ ID NO: 30 - DNA, 29 bp, Artificial
  Primer
aatgatgagaacctgatcgcatagtggtg

SEQ ID NO: 31 - DNA, 27 bp, Artificial
  Primer
aatgatgagaacctgagcatagtggtg

SEQ ID NO: 32 - DNA, 28 bp, Artificial
  Primer
aatgatgagaacctgacgcatagtggtg

SEQ ID NO: 33 - DNA, 26 bp, Artificial
  Primer
ggactctgtacctcctatccacgtca

SEQ ID NO: 34 - DNA, 27 bp, Artificial
  Primer
ccctcggcccctgcactgtttcccaga

SEQ ID NO: 35 - DNA, 19 bp, Artificial
  Primer
gtttcccagggcttatccc

SEQ ID NO: 36 - DNA, 17 bp, Artificial
  Primer
gtttcccagcttatccc

SEQ ID NO: 37 - DNA, 20 bp, Artificial
  Primer
gcatcgatgaagaacgcagc

SEQ ID NO: 38 - DNA, 20 bp, Artificial
  Primer
tcctccgcttattgatatgc

SEQ ID NO: 39 - DNA, 24 bp, Artificial
  Primer
cggtcctcgagcgtatggggcttt

SEQ ID NO: 40 - DNA, 26 bp, Artificial
  Primer
ccgacgcattttttgtgcaacttgtt

SEQ ID NO: 41 - DNA, 26 bp, Artificial
  Primer
ccgaacgcatttatttgcaacttgtt

SEQ ID NO: 42 - DNA, 25 bp, Artificial
  Primer
ccgacgcatttgtttgcaacttgtt

SEQ ID NO: 43 - DNA, 25 bp, Artificial
  Primer
ccgacgcatttatttgcaacttgtt

SEQ ID NO: 44 - DNA, 24 bp, Artificial
  Primer
gccgacgttttccaaccattcttt

SEQ ID NO: 45 - DNA, 23 bp, Artificial
  Primer
gccgacgttatccaaccattttt

SEQ ID NO: 46 - DNA, 24 bp, Artificial
  Primer
cagccgacacccaactttattttt

SEQ ID NO: 47 - DNA, 24 bp, Artificial
  Primer
ccagccgacacccaactttatttt

SEQ ID NO: 48 - DNA, 24 bp, Artificial
  Primer
gccagccgacacccaactttattt

SEQ ID NO: 49 - DNA, 25 bp, Artificial
  Primer
ccgaacgcaaatcaatctttttcca

SEQ ID NO: 50 - DNA, 25 bp, Artificial
  Primer
cttgccgaacgcaaatcaatctttt

SEQ ID NO: 51 - DNA, 24 bp, Artificial
  Primer
gccgaacgcaaatcaatctttttc

SEQ ID NO: 52 - DNA, 24 bp, Artificial
  Primer
ttcggactctttgatgattcataa

SEQ ID NO: 53 - DNA, 23 bp, Artificial
  Primer
tcttcggactctttgatgattca

SEQ ID NO: 54 - DNA, 22 bp, Artificial
  Primer
ggaaggtgttgtggttaatacc

SEQ ID NO: 55 - DNA, 22 bp, Artificial
  Primer
ggaaggtgttgtggttaataac

SEQ ID NO: 56 - DNA, 20 bp, Artificial
  Primer
caaatgttgggtgaacggct

SEQ ID NO: 57 - DNA, 22 bp, Artificial
  Primer
aaagcaaatgttgggtgaacgg

SEQ ID NO: 58 - DNA, 24 bp, Artificial
  Primer
ttctcgatggtccataccttagat

SEQ ID NO: 59 - DNA, 24 bp, Artificial
  Primer
tagatgcgttagcattaatcaggc

SEQ ID NO: 60 - DNA, 24 bp, Artificial
  Primer
ttagatgcgttagcattaatcagg

SEQ ID NO: 61 - DNA, 25 bp, Artificial
  Primer
cttagatgcgttagcattaatcagg

SEQ ID NO: 62 - DNA, 23 bp, Artificial
  Primer
atggtccataccttagatgcgtt

SEQ ID NO: 63 - DNA, 22 bp, Artificial
  Primer
aggccttcgggttgtaaagtac

SEQ ID NO: 64 - DNA, 22 bp, Artificial
  Primer
ggaagggagtaaagttaatacc

SEQ ID NO: 65 - DNA, 22 bp, Artificial
  Primer
gaacaagtgccgttcgaatagg

SEQ ID NO: 66 - DNA, 22 bp, Artificial
  Primer
gaacaagtaccgttcgaatagg

SEQ ID NO: 67 - DNA, 22 bp, Artificial
  Primer
aggttttcggatcgtaaagctc

SEQ ID NO: 68 - DNA, 20 bp, Artificial
  Primer
aacgatcggaggaccgaagg

SEQ ID NO: 69 - DNA, 21 bp, Artificial
  Primer
acaacgatcggaggaccgaag

SEQ ID NO: 70 - DNA, 22 bp, Artificial
  Primer
gaacaagtgctagttgaataag

SEQ ID NO: 71 - DNA, 22 bp, Artificial
  Primer
aggctttcgggtcgtaaaactc

SEQ ID NO: 72 - DNA, 20 bp, Artificial
  Primer
tccggttgagggctctatct

SEQ ID NO: 73 - DNA, 19 bp, Artificial
  Primer
caactggcgcgtacgtaaa

(19) United States
(12) Patent Application Publication
(10) Pub. No.: US 2005/0266466 A1
(43) Pub. Date: Dec. 1, 2005

(54) Microarray-based method for amplifying and detecting nucleic acids during a continuous process

(75) Inventors: Eugen Ermantraut, Jena (DE); Ralf Bickel, Jena (DE); Thomas Ellinger, Jena (DE); Annette Wagenhaus, Jena (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Clondiag Chip Technologies GmbH, Jena (DE)

(21) Appl. No.: 11/132,971

(22) Filed: May 19, 2005

Related U.S. Application Data
(63) Continuation of application No. PCT/EP03/12905, filed on Nov. 18, 2003.

(30) Foreign Application Priority Data
Nov. 19, 2002 (DE) 102 53 966.9

(51) Int. Cl. C12Q 1/68; C12P 19/34

(52) U.S. Cl. 435/6; 435/91.2

(57) ABSTRACT

The invention concerns methods, in the context of slide-based evaluation, for efficiency amplifying and detecting nucleic acids during a continuous process. The invention mainly concerns methods which consist in performing a polymerase chain reaction occurs, whereto a competitor element is added at the beginning of the reaction. The invention also concerns methods which consist in an hybridization in the presence of a molecule which, proximate to the hybridizing sequences, is bound to one of the hybridizing nucleic acid molecules

### CROSS-REFERENCE TO RELATED APPLICATIONS

This application is a continuation of PCT/EP03/12905, filed Nov. 18, 2003, which claims the benefit under § 119 of DE 102 53 966.9, filed Nov. 19, 2002, the contents of both of which are hereby incorporated by reference.

### BACKGROUND OF THE INVENTION

Biomedical tests are often based on the detection of an interaction between a molecule, which is present in known amount and position (the molecular probe), and an unknown molecule or unknown molecules, respectively, that is or are to be detected (the molecular target molecules). In modern tests, the probes are applied on supports, the so-called microarrays or chips, in the form of a compound library, so that one sample can be analyzed parallel at several probes simultaneously (Lockhart et al. (2000) Nature, 405, 827-836). For the production of the microarrays, the probes are herein usually immobilized on a suitable matrix in a predetermined manner, as for example is described in WO 00/12575 (see e.g. U.S. Pat. No. 5,412,087, WO 98/36827), or are produced synthetically (see e.g. U.S. Pat. No. 5,143,854), respectively.

Herein, the detection of an interaction between the probe and the target molecule is performed as follows:

The probe or the probes, respectively, are fixed in a predetermined manner to a certain matrix in the form of a microarray. The targets are then brought into contact with the probes in a solution and incubated under defined conditions. As a result of the incubation, a specific interaction takes place between the probe and the target. The bond occurring herein is substantially more stable than the bond of the target molecules to probes which are not specific for the target molecule. For removing those target molecules that have not been specifically bound, the system is washed with corresponding solutions or is heated.

The detection of the specific interaction between a target and its probe can then be achieved by means of a variety of methods, which usually depend on the type of the marker, which has been introduced into the target molecules before, during, or after the interaction of the target molecules with the probes. Typically, such markers are fluorescent groups, so that specific target-probe interactions can be read out fluorescence-optically with a high local resolution and in an uncomplicated way in comparison to other common detection methods (esp. mass-sensitive methods) (Marshall et al. (1998) Nature Biotechnology, 16, 27-31; Ramsay (1998) Nature Biotechnology, 16, 40-44).

Depending on the compound library immobilized on the microarray and on the chemical nature of the target molecules, interactions between nucleic acids and nucleic acids, between proteins and proteins, and between nucleic acids and proteins can be examined with the help of this test principle (for survey see Lottspeich et al. (1998) Bioanalytik, Spektrum Akademischer Verlag, Heidelberg Berlin).

Herein, antibody libraries, receptor libraries, peptide libraries, and nucleic acid libraries can be used as compound libraries immobilized on microarrays or chips. The nucleic acid libraries play the most important role by far.

These are microarrays, on which deoxyribonucleic acid (DNA) molecules, ribonucleic acid (RNA) molecules or molecules of nucleic acid analogs (e.g. PNA) are immobilized. It is prerequisite for the bond of a target molecule (DNA molecule or RNA molecule) labeled with a fluorescence group to a nucleic acid probe of the microarray that both the target molecule and the probe molecule are present in the form of a single-stranded nucleic acid.

An efficient and specific hybridization can only take place between such molecules. Single-stranded nucleic acid target molecules and nucleic acid probe molecules can normally be obtained by means of heat denaturation and parameters to be selected optimally (temperature, ionic strength, concentration of helix-destabilizing molecules), which ensures that only probes with virtually perfectly complementary (corresponding to each other) sequences remain paired with the target sequence (Leitch et al. (1994) In vitro Hybridisierung, Spektrum Akademischer Verlag, Heidelberg Berlin Oxford).

A typical example for the use of microarrays in biological test methods is the detection of microorganisms in samples in biomedical diagnostics. Herein, it is taken advantage of the fact that the genes for ribosomal RNA (rRNA) are dispersed ubiquitously and have sequence portions, which are characteristic for the respective species. These species-characteristic sequences are applied on a microarray in the form of single-stranded DNA oligonucleotide probes. The target DNA molecules to be examined are first isolated from the sample to be examined and equipped with fluorescent markers. Subsequently, the labeled target DNA molecules are incubated in a solution with the probes fixed on the microarray; unspecifically occurring interactions are removed by means of corresponding washing steps and specific interactions are detected by means of fluorescence-optical evaluation. In this manner, it is possible to detect e.g. several microorganisms simultaneously in one sample by means of one single test. In this test method, the number of detectable microorganisms theoretically only depends on the number of the specific probes, which have been applied on the microarray.

A further example for a use in a medical test method is the production of a single nucleotide polymorphism (short: SNP) profile as starting point for an individualized therapy.

The entirety of the genetic information of a creature is individual in all creatures, with the exception of identical (multiple) twins and clones. Herein, the extent of dissimilarity increases as the degree of biological relationship among the individuals decreases. Here, the so-called single nucleotide polymorphisms (SNPs) are the most common variation in the human genome.

On the basis of the respective SNP profile of a human, it shall now become possible to assess which individual responds to the respective medicines or which individual is likely to exhibit unwanted side effects in medicinal therapy. Figures from the field of oncology show that often only 20 to 30% of the patients respond to specific active agents. The remaining 70% are often unable to utilize them due to obviously genetic reasons. Herein, test systems based on DNA-arrays are helpful in making decisions and represent an excellent method for testing the patient quickly and reliably and by means of a few simple steps for few SNPs exclusively relevant for a disease. Thereby, therapies can be varied individually for patients.

In many tests in biomedical diagnostics, the problem arises that first of all, before the beginning of the actual test method, the target molecules have to be present in sufficient form and therefore often have to be amplified from the sample first. The amplification of DNA molecules is achieved by means of the polymerase chain reaction (PCR). For the amplification of RNA, the RNA molecules have to be transformed into correspondingly complementary DNA (cDNA) by means of reverse transcription. This cDNA can then also be multiplied (amplified) by means of PCR. PCR is a standard laboratory method (Sambrook et al. (2001) Molecular Cloning: A laboratory manual, 3rd edition, Cold Spring Harbor, N.Y., Cold Spring Harbor Laboratory Press).

The amplification of DNA by means of PCR is relatively fast, allows a high throughput of samples in small batch volumes by means of miniaturized methods, and is efficient in operation due to automation.

However, a characterization of nucleic acids by means of mere amplification is not possible. It is rather necessary to apply analysis methods like nucleic acid sequence identification, hybridization, and/or electrophoretic separation and isolation methods for the characterization of the PCR products subsequently to the amplification.

Generally, devices and methods for the amplification of nucleic acids and their detection should be designed in such a way that as few interventions as possible by the experimenter are necessary. The advantages of methods allowing an amplification of nucleic acids and their detection, and in the course of which the experimenter has to intervene only minimally, are obvious. On the one hand, contaminations are avoided. On the other hand, the reproducibility of such methods is substantially increased, as they are accessible to automation. This is also extremely important considering admission of diagnostic methods.

At present, there are a multiplicity of methods for the amplification of nucleic acids and their detection, in which first the target material is amplified by means of PCR amplification and subsequently the identity and the genetic state, respectively, of the target sequences is determined by means of hybridization against a probe array. Normally, the amplification of the nucleic acid molecules or the target molecules to be detected, respectively, is necessary in order to have at one's disposal amounts sufficient for a qualitative and quantitative detection by means of hybridization.

Both the PCR amplification of nucleic acids and the detection of the same by means of hybridization are subject to several elementary problems. In the same manner, this applies to methods combining a PCR amplification of nucleic acids and their detection by means of hybridization.

One of the problems arising in methods combining PCR and hybridization is based on the double-strandedness of the target molecules. Assuming a double-stranded template molecule, classical PCR amplification reactions usually produce double-stranded DNA molecules. These can only hybridize with the probes of the probe array after previous denaturation. During the hybridization reaction, the very rapid formation of double strands in the solution competes against the hybridization with the immobilized probes of the probe array. The intensity of the hybridization signals, and therefore the quantitative and qualitative evaluation of the results of the method, is strongly limited by this competition reaction.

In addition, there are those problems based on the hybridization reaction per se and on the probes and targets made to hybridize, respectively. PCR products used as targets for array hybridization reactions normally have a length of at least about 60 base pairs. This corresponds to the sum of the lengths of the forward and reverse primers used for the PCR reaction as well as to the region, which is amplified by the PCR and which exhibits complementarity to the probe on the array. Single-stranded molecules of this length are seldom present in solution in an unstructured form, i.e. linearly stretched, but are of more or less stable secondary structures like e.g. hairpins or other helical structures. If these secondary structures affect the target region, which exhibits complementarity to the probe, the formation of said secondary structures prevents an efficient hybridization of the target to the probe. Therefore, the formation of secondary structures can also inhibit an efficient hybridization and impede, if not even prevent, a quantitative and qualitative evaluation of the results of the method.

In the prior art, it is attempted to meet the problems described with a special process management and special protocol steps, respectively. These are, inter alia, selecting certain PCR conditions, processing and special treatment of the PCR amplification products and variation and alteration of the reaction parameters from PCR to hybridization.

Herein, the person skilled in the art has at his disposal several known working points. For example, the efficiency of the hybridization of double-stranded target molecules to probe arrays can be improved by a buffer exchange. Classical hybridization buffers have a high ionic strength and are optimized to such an effect as to minimize the competition of the two above-mentioned effects, i.e. the competition of the hybridization of the individual strands of the target against the hybridization to the probe of the array and the formation of secondary structures. Contrarily, PCR buffers normally have a low ionic strength and are correspondingly not optimized for array hybridizations to probe arrays. Therefore, hybridizations of double-stranded PCR fragments at probe arrays are not performed directly in the PCR buffer, but rather after replacing it with a hybridization buffer. Here, the buffer exchange often takes place subsequently to the alcohol precipitation of the PCR product or subsequently to purification by means of affinity columns known in the prior art (e.g. Qiaquick PCR purification kit by Qiagen, Hilden, Germany), respectively. A typical protocol for a method, wherein PCR products are detected by means of hybridization to probe arrays, can be taken from the GeneChip P450 Kit by Affymetrix, Inc. (Santa Clara, Calif., USA). The disadvantage of said method is that the buffer exchange indicates an additional working step and therefore, as explained above, there is the danger of an additional contamination in the course of the working process.

Another possibility of counteracting an inefficient hybridization owing to the competition when hybridizing or owing to a formation of secondary structures in the target lies in the efficient labeling of the target. The negative influence of the double-strand formation of the target in the solution and the formation of secondary structures on the intensity of the hybridization signal can partially be compensated by an efficient labeling of the PCR product, e.g. by application of labeled deoxynucleotide triphosphates.

Beside the higher costs in comparison with a final labeling by means of applying labeled primers, this method has the additional disadvantage that a detachment of the labeled components from the PCR product is necessary before the hybridization in order to keep the unspecific background signal within a justifiable limit during the subsequent hybridization. This additional step complicates the detection method and is an additional contamination source. Furthermore, this additional working step prevents the development of a continuous analysis process, which combines PCR amplification and array hybridization and which does not have to be influenced by the experimenter.

A further approach to solve said problems, which lead to an inefficient hybridization due to the formation of secondary structures, is the fragmentation of the PCR amplification product. The PCR product can be fragmented into short DNA portions usually comprising 20 to 40 bases by means of enzymatic or chemical methods, so that the formation of stable secondary structures is prevented. Furthermore, as the surfaces of probe arrays usually offer a better accessibility for these short fragments, the competition of the double strand formation of the target in the solution against the hybridization against the immobilized probes on the probe array is minimized by the fragmentation of the PCR product.

However, this method has several disadvantages. On the one hand, the fragmentation of the DNA is an additional protocol step requiring at least one additional buffer exchange. The disadvantages connected therewith have already been stated above. Furthermore, the fragmentation of the DNA does usually not take place in a sequence-specific manner, so that a part of the DNA molecules is fragmented in the region, which exhibits complementarity to the probe. Targets fragmented in such a way naturally bind worse or not at all to the immobilized probes. In order to prevent the probes from being fragmented, the enzymes or chemical substances used for the fragmentation need to be inactivated beforehand. This can also lead to additional working steps. Furthermore, the fragmentation excludes a cost-efficient and easy-to-perform final labeling of the PCR products, as internal fragments are only detectable in case of internal labeling after the fragmentation.

Owing to the disadvantages stated above, as occurring with the hybridization of targets amplified by means of classical PCR protocols to immobilized probes of a probe array, attempts have been made in the prior art to modify the PCR conditions to such an effect that during the PCR only one of the two strands of the double-stranded DNA template used for the PCR is amplified.

The competition of the double strand formation of the target molecules against the hybridization with the probe molecules during hybridization should in principle be prevented by means of the development of PCR protocols, which allow the production of a single-strand surplus.

In the case of the PCR, single-stranded DNA can either be produced subsequently to the reaction from the double-stranded PCR product, or PCR conditions are selected, wherein one of the strands is produced in surplus in the course of the PCR.

For the production of single-stranded DNA target molecules from double-stranded PCR products, one of the two strands can be enzymatically degraded in a selective manner, while the other strand is resistant to the nucleolytic degradation and thereby is maintained.

If, for example, one of the used primers is modified with several phosphothioate components at its 5′-end during the PCR, the obtained PCR product can be treated with T7 gene 6 exonuclease. Herein, single-stranded DNA is produced, as only the non-modified strand can be degraded exonucleolytically, while the phosphothioate-modified strand is resistant to the exonucleolytic degradation (Nikiforov et al. (1994) PCR Methods Appl., April 3 (5), 285-291).

In an alternative method, one of the two primers used for PCR is modified with a phosphate group at its 5′-end. When subsequently treating the PCR fragment with lambda exonucleases, only the phosphorylated strand is degraded exonucleolytically. The other strand is maintained as single strand (Michel et al. (1997) Histochem. J., 29(9), 685-683; Kujau et al. (1997) Mol. Biotechnol. 3, 333-335; Null et al. (2000) Analyst, 125 (4), 619-626).

Alternatively, double-stranded PCR products can be transformed to single-stranded DNA by means of detaching one strand and be used as target molecules for hybridization. For example, one of the primers used for PCR can be labeled with biotin. If the amplified DNA double strand is subsequently bound to a fixed carrier coated with streptavidin, the streptavidin-coated carrier can, after denaturation, be separated from the solution by means of e.g. centrifugation, so that one of the strands remains in the solution (Bowman et al. (1993) Methods Enzymol., 224, 339-406).

All of the mentioned methods have the disadvantage that the detachment of one of the two strands amplified within the scope of the PCR is an additional working step and correspondingly brings about the above-mentioned disadvantages. Furthermore, the phosphothioates used for the production of the primers and the biotin markers used for labeling of the primers, respectively, are connected with increased costs.

Methods, wherein a single strand surplus already emerges during the PCR reaction, are generally known as linear or asymmetric PCR.

In a classical linear PCR reaction, a double-stranded template is amplified in a PCR reaction, to which only one single primer was added. Correspondingly, on the basis of this primer, only one of the two strands is produced, which is then present in a surplus (Kaltenboeck et al. (1992) Biotechniques, 12(2), 164-166). However, the linear PCR has the disadvantage that the target amounts it amplifies are normally not sufficient for an efficient quantitative and qualitative detection by means of hybridization of a probe array.

Generally, a PCR is called symmetrical, if both primers are present in identical molar amounts. The amplification rate then follows exponential kinetics of the form 2ⁿ (n=number of PCR cycles). The combination of a symmetrical PCR reaction with an amount of primers limited with respect to the template and subsequent addition of one single primer in surplus followed by a linear amplification is usually described as two-step linear or asymmetrical PCR. It is to be seen as the disadvantage of this method that the primer for performing the linear PCR is added only after performing the symmetrical PCR, which represents an additional intervention in the experimental system.

Another method of the two-step asymmetrical PCR is described in U.S. Pat. No. 5,891,625. Subsequently to a symmetrical PCR amplification, peptide nucleic acids (PNA) exhibiting complementarity to one of the two primers are added to the reaction. Due to hybridization of the corresponding primer with the antisense PNA, only the other primer remains available for the further PCR amplification, so that only one strand is amplified linearly. In turn, the disadvantage of this method is that the antisense PNA is added to the reaction only after performance of a symmetrical PCR, which represents an additional manipulating step.

U.S. Pat. No. 5,627,054 describes a further method of the two-step asymmetrical PCR, wherein subsequently to performing a symmetrical PCR a competitor is added to the reaction, which blocks the binding sites of one of the primers on the template. The competitor is a nucleic acid, which cannot be extended to form the polymerase used for PCR due to its sequence or a chemical modification at its 3′-end. Usually, the competitor is added in molar surplus in relation to the primer. Here, the competitor is also added to the reaction only after performing a symmetrical PCR. This additional working step is accepted because the amount of single strand produced when omitting the symmetrical PCR is not sufficient to be efficiently detected in a quantitative and qualitative hybridization on a probe array.

As already depicted, the methods described have in common the disadvantage that at least one additional reaction step, which requires an intervention by the experimenter, is necessary for the production of single-stranded DNA. Therefore, these methods are not suitable for the application in closed systems or on fluctuation thermocyclers based on meandering channels, respectively (Köhler et al. (1998) Micro Channel Reactors for Fast Thermocycling, Proc. 2^{nd} International Conference on Microreaction Technology, New Orleans, 241-247), wherein an addition or a detachment of components during the reaction is not possible.

Alternative methods allow the asymmetrical PCR amplification in one step. For example, a strand can be produced in surplus by addition of the primers in an asymmetrical ratio (i.e. one of the primers is present in a smaller molar amount and is consumed in the course of the reaction). However, the disadvantage of this method is that a single strand surplus is achieved only after reaching a certain concentration of the PCR product. However, achieving this concentration in turn strongly depends on the initial concentration of the template, so that the critical concentration possibly is not even achieved in samples with a small amount of template. The amplification product would then only be present in double-stranded form and the signal in case of a hybridization-based analysis would be weakened disproportionately due to the lowly concentrated samples.

As has already been depicted above, the formation of secondary structures in the target is of substantial influence on the strength of the hybridization signal (Southern et al. (1994) Nucleic Acids Res., 22, 1368-1373; Sohail et al. (1999) RNA, 5, 646-655).

Correspondingly, different attempts have been made to increase the signal intensities in hybridization detection methods by means of reduction of the secondary structure of the target molecules.

For example, N4-ethyl-substituted cytosine moieties, which strongly reduced the formation of secondary structures, were used during the chemical synthesis of a DNA molecule (Nguyen et al. (2000) Nucleic Acids Res., 28, 3904-3909). However, this method cannot be applied, if the target molecules to be detected are not chemically, but enzymatically synthesized.

A further possibility of meeting the weakening of signal intensities in hybridization detection methods due to formation of secondary structures is the use of the already mentioned PNAs as probes. Due to the physicochemical properties of the PNAs, the hybridization can then be performed at comparatively low ionic strengths, so that the proneness of target molecules to form secondary structures is minimized. However, PNA arrays are not commercially available at present, so that said method, although it is conceivable, is not practicable in standard applications.

The hybridization of probes with target molecules can also be stabilized and the signal intensities can be increased correspondingly, if the target molecules are hybridized with oligonucleotide probes, which are located in the immediate proximity of the sequence hybridizing with the probe of the probe array, before the hybridization on the array. When subsequently hybridizing the “pre-hybridized” target molecule with the probe of the probe array, in this case a continuous helix is built covering the region of the probe and of the previously hybridized oligonucleotide, so that the total hybrid is stabilized via so-called ‘stacking interactions’ (O'Meara et al. (1998) Anal. Biochem., 225(2), 195-203; Maldonado-Rodriguez et al. (1999) Mol. Biotechnol., 11(1), 1-12). Possibly, destabilization of secondary structures in the target molecule also plays a role in hybridization stabilization and corresponding increase of signal intensity.

However, it is disadvantageous that the intensity of the hybridization signal is drastically reduced when the stabilization caused by ‘stacking interactions’ is omitted by means of inserting a gap of one or more bases between the binding sites of the pre-hybridized oligonucleotide and the probe of the probe array. In this respect, it is very time-consuming to select the pre-hybridized oligonucleotides in an exact manner. It is a further disadvantage that the addition of the oligonucleotides is not possible as early as at the beginning of the PCR amplification, as otherwise they would be used as PCR primers and would lead to additional PCR fragments. Furthermore, the oligonucleotides lose their function as secondary structure breakers due to the enzymatic extension reaching into the target region. As an initial addition of the oligonucleotides to the PCR is not possible, no continuous process involving PCR and hybridization can be established. Moreover, additional process steps are required, which brings about the disadvantages already depicted several times.

From the methods depicted above it follows, that at present there is no method allowing the array-based performance of analyses, which are based on an amplification of the target material by means of PCR and subsequent analysis of the amplification product by means of hybridization against probe arrays in a continuous process, while maintaining high sensitivity and specificity.

Such a method should allow combining the reaction setup necessary for the entire course including array at the beginning of the analysis, performing PCR and hybridization as a continuous process, and subsequently reading out the hybridization pattern on the probe array. Such a simple, sensitive, and specific method would be most desirable not only due to reasons of cost and time saving. Furthermore, it is prerequisite for the performance of sensitive and specific combined amplification and hybridization reactions in array format in closed reaction chambers. Therewith, such a method would represent an important contribution on the way to establishing ‘point of care’ diagnostic methods.

Likewise, at present there is no array-based method allowing a parallel real-time quantification of targets by means of PCR amplification and hybridization. Such a method could be realized for example with alternating PCR amplification and hybridization steps, wherein subsequently to each amplification step a hybridization against an array takes place and the PCR reaction is quantified on the basis of the hybridization signals. In comparison with the prior art, such a method would demand that on the one hand PCR and hybridization are performed in a continuous process and that on the other hand a very high sensitivity in hybridization-based detection in the entire course of the PCR amplification can be achieved.

### SUMMARY OF THE INVENTION

It is therefore an object of the present invention to provide microarray-based methods, which allow a PCR amplification of the target material and its detection by means of hybridization against probe arrays at high sensitivity and specificity in a continuous process. In particular, it is an object of the present invention to provide methods for microarray-based evaluations, wherein the PCR amplification of the target material and the detection of the amplified material can be performed by means of hybridization against a probe array without interposed experimental steps or processing reactions. In particular, it is an object of the present invention to provide methods for microarray-based evaluations, wherein the target material is produced in a continuous PCR amplification process yielding a single strand surplus sufficient for the hybridization.

It is a further object of the invention to provide a method for parallel hybridization-based real-time quantification of PCR targets, which is based on the increase of sensitivity of the hybridization reaction and the performance of the PCR amplification and hybridization reaction in a continuous, cyclic process.

It is also an object of the invention to provide a method, wherein in an asymmetrical PCR an amount of single strand surplus is amplified, which is sufficient for an efficient detection in a hybridization method.

Besides, it is an object of the present invention to provide methods for the microarray-based evaluation, wherein the formation of secondary structures of the target material is prevented.

These and other objects of the present invention, as resulting from the description, are attained by providing the subject matter of the independent claim. Preferred embodiments of the invention are defined by the subclaims.

One aspect of the present invention is a method for the efficient amplification of nucleic acids and their detection in a continuous process, which is characterized in that the nucleic acid to be detected is first amplified by means of a PCR in single strand surplus, wherein at least one competitor, which inhibits the formation of one of the two PCR-amplified template strands, is added to the reaction at the beginning, and the amplified nucleic acid is detected by means of hybridization with a complementary probe.

It has surprisingly been found within the scope of the present invention that, by means of a PCR reaction, to which a competitor, which inhibits the amplification of one of the two template strands, was added at the beginning, a sufficient amount of single-stranded nucleic acid molecules are amplified in a surplus in order to detect these in a second step by means of hybridization with a complementary probe.

On the basis of theoretical considerations it was to be assumed that with the amounts of competitors employed according to the present invention, the amplification of a template strand is substantially inhibited, which leads to an almost linear amplification. Although in this manner a single strand surplus could be achieved in the PCR, it would not suffice for an efficient hybridization owing to small amounts.

However, it was surprisingly observed that although the exponential amplification of the template strands that takes place is retarded owing to the competitor, it still yields an amount of single strands large enough to guarantee for an efficient hybridization. In particular, the loss of amount is more than compensated by the single strand surplus in hybridization-based assays.

Up to now it had been assumed that the sole performance of an asymmetrical PCR reaction does not amplify an amount of single-stranded nucleic acid molecules large enough to be detected in subsequent detection reactions like, e.g., a hybridization with a corresponding probe. Therefore, a two-step asymmetrical or linear PCR had as yet always been performed in the prior art for the amplification of single-stranded nucleic acid molecules, i.e. a classical symmetrical PCR amplification was performed before the asymmetrical or linear PCR amplification, which meant, however, a multiple experimental intervention, like e.g. the addition of new primers, in the course of the process management.

Contrarily to this, the present invention surprisingly shows that by means of addition of competitors to the PCR reaction setup at the beginning of the amplification reaction an efficient amplification of the target sequence can be achieved simultaneously with a single strand surplus that is large enough to allow an efficient, quantitative and qualitative detection of the amplified single strands by means of hybridization with corresponding probes.

Therefore, one aspect of the invention is a method for the efficient amplification of nucleic acids, wherein the nucleic acid, which is to be detected subsequently, if necessary, is amplified by means of a PCR, wherein at least one competitor, which inhibits the formation of one of the two template strands amplified or amplifiable, respectively, by means of the PCR, is added to the reaction at the beginning.

In a preferred embodiment of the invention, the amplified nucleic acids are detected by means of subsequent hybridization with a complementary probe.

A further aspect of the present invention is a method for the efficient amplification of nucleic acid molecules and their detection by means of hybridization in a continuous process, wherein
a) the nucleic acid to be detected is first amplified by means of a PCR reaction and at least one competitor, which inhibits the formation of one of the two template strands amplified by means of the PCR, is added to the reaction at the beginning, and
b) the amplified nucleic acid molecule is detected by means of hybridization with a complementary probe.

In particular, one aspect of the present invention is a method for the efficient amplification of nucleic acid molecules and their detection by means of hybridization in a continuous process, which is characterized in that a DNA molecule, which competes against one of the primers used for the PCR amplification of the template for binding to the template and which is enzymatically not extendable, is added to the PCR. The single-stranded nucleic acid molecules amplified by means of the PCR are then detected by means of hybridization with a complementary probe.

A further aspect of the invention is a method for the amplification of nucleic acid molecules and their detection by means of hybridization in a continuous process, which is characterized in that a nucleic acid to be detected is first amplified in single strand surplus by means of a PCR and is detected by a subsequent hybridization with a complementary probe, wherein a competitor, which is a DNA molecule or a molecule of a nucleic acid analog capable of hybridizing to one of the two strands of the template but not to the region detected by means of probe hybridization and which cannot be extended enzymatically, is added to the PCR reaction at the beginning.

A further aspect of the invention is the use of the method according to the present invention for microarray-based experiments and diagnostic methods.

In one aspect of the present invention, in the method for the efficient amplification of at least one template nucleic acid, the nucleic acid is amplified by means of a PCR, wherein at least one competitor, which inhibits the formation of one of the two template strands amplified by means of the PCR, is added to the reaction at the beginning.

Preferably, the competitor is selected from proteins, peptides, intercalators, aptamers, nucleic acids, and/or nucleic acid analogs. The competitor nucleic acid is selected from DNA and/or RNA molecules. The competitor nucleic acid analog is preferably selected from PNA, LNA, TNA, and/or a nucleic acid analog having a phosphothioate bond.

Preferably, the competitor is a nucleic acid and/or a nucleic acid analog, wherein the competitor is enzymatically not extendable. In particular, the 3′-end of the nucleic acid and/or the nucleic acid analog is enzymatically not extendable, particularly preferably due to the fact that it has no free 3′-OH group and, for example, has instead of the 3′-OH group at its 3′-end an amino group, a phosphate group, a biotin moiety, a fluorophor or a fluorescent colorant, respectively, or a hydrogen atom. Suitable fluorescent colorants are for example colorants normally used for labeling primers or monomers.

It is further preferred that a competitor nucleic acid or a competitor nucleic acid analog, respectively, is used, which has at its 3′-end at least one, two, three, four, or five nucleotides or nucleotide analogs, which are not complementary to the corresponding positions in the respective template strand.

Preferably, the competitor inhibits the formation of one of the two template strands amplified by means of the PCR by binding to one of the primers used in the PCR and/or by binding to one of the template strands.

In one embodiment, it is preferred that the competitor binds to one of the primers used in the PCR. Thereby, the hybridization of the primer with a template strand is inhibited.

Preferably, the competitor is a nucleic acid and/or a nucleic acid analog with a sequence that is at least in part complementary to one of the primers used in the PCR.

It is further preferred that the competitor is linked covalently with the primer. The covalent link between competitor and primer does preferably not serve as template strand for polymerases.

Preferably, the stability of the complex of primer with competitor is lower than the stability of the complex of the primer with its specific primer binding site on the template strand. Concerning the temperature regime, the amplification is particularly preferably performed as a two-phase process, wherein during a first phase an annealing temperature is selected, at which the primer binds to its specific primer binding site on the template strand but not to the competitor, and during a second phase an annealing temperature is selected, at which also a bond between primer and competitor takes place.

In a further embodiment, it is preferred that the competitor binds one of the template strands, which inhibits its amplification.

In particular, the competitor competes for binding to one of the template strands against one of the primers used for the PCR. Herein, it is further preferred that the stability of the complex of the competitor with the specific primer binding site of the primer on the template is lower than the stability of the complex of the primer with its specific primer binding site on the template strand. Concerning the temperature regime, the amplification is particularly preferably performed as a two-phase process, wherein during a first phase an annealing temperature is selected, at which the primer, but not the competitor, binds to the specific binding site of the primer on the template strand, and during a second phase an annealing temperature is selected, at which a bond of the competitor to the specific binding site of the primer on the template also takes place.

Preferably, the competitor simultaneously acts as a secondary structure breaker.

It is further preferred that several competitors are employed.

Preferably, at least one of the competitors exclusively acts as a secondary structure breaker.

Preferably, the complex of the competitor acting exclusively as a secondary structure breaker with the template strand is less stable than the complex of at least one competitor acting in an inhibiting manner with the template strand. Concerning the temperature regime, the amplification is particularly preferably performed as a two-phase process, wherein during a first phase an annealing temperature is selected, at which the competitor acting in an inhibiting manner, but not the competitor acting exclusively as a secondary structure breaker, binds to a template strand, and during a second phase a temperature is selected, at which the competitor acting exclusively as a secondary structure breaker also binds to the template strand.

It is further preferred that the competitor and/or the secondary structure breaker is a nucleic acid and/or a nucleic acid analog comprising at least 10, preferably at least 15, particularly preferably at least 17 and most preferably at least 18 nucleotides. Likewise, it can be preferred that the competitor and/or the secondary structure breaker is a nucleic acid and/or a nucleic acid analog comprising at least 10, preferably at least 15, particularly preferably at least 20, also particularly preferably at least 25, in particular preferably at least 30 nucleotides.

Further, the competitor and/or secondary structure breaker are preferably present at the beginning of the reaction in at least equimolar amounts relative to the target and/or to one of the PCR primers, preferably in a molar surplus relative to the target and/or to one of the PCR primers. Particularly preferably, the ratio of primer or target, respectively, and competitor or secondary structure breaker, respectively, relative to the molar amounts lies between 0.01 and 0.99, preferably between 0.05 and 0.8 and particularly preferably between 0.1 and 0.5 at the beginning of the reaction.

DNA is preferably employed as template nucleic acid. Likewise, RNA can be employed as template nucleic acid and be transcribed to DNA by means of a reverse transcriptase before the PCR amplification.

Furthermore, in one embodiment the PCR is a multiplex PCR.

A further aspect of the present invention relates to a method for the detection of at least one nucleic acid, wherein the nucleic acid is amplified by means of an amplification method according to the present invention, as is described above, and are subsequently detected with a complementary probe by means of hybridization.

Preferably, the amplification of the nucleic acids and their detection by means of hybridization against a probe are performed in a continuous process.

It is further preferred that the competitor binds the template strand complementary to the probe. Particularly preferably, the competitor binds the template strand complementary to the probe in a region, which is not addressed by the probe.

It is further preferred that the competitor binds in the immediate proximity of the sequence region addressed by the probe. Particularly preferably, the competitor is a DNA oligonucleotide hybridizing with the template strand, which is detected by the hybridization, in immediate proximity to the sequence region addressed by the probe.

Furthermore, it can be preferred that the competitor binds near the sequence region addressed by the probe. Here, it is also particularly preferred that the competitor is a DNA oligonucleotide hybridizing with the template strand, which is detected by means of the hybridization, near the sequence region addressed by the probe.

It is further preferred that at the beginning a competitor, which competes for binding to the template against one of the PCR primers, and at least one competitor binding to the target near or in immediate proximity to the sequence detected by the probe and thus acting as a secondary structure breaker is added to the PCR.

Furthermore, it can be preferred that at least one of the competitors acting exclusively as secondary structure breaker is added to the reaction mixture only after completion of the PCR.

Furthermore, PCR and hybridization can preferably be performed in the same buffer system.

Furthermore, PCR and/or hybridization are preferably performed in a closed reaction chamber. Such a closed reaction chamber is for example described in the international patent applications WO 01/02094, WO 03/031063, and WO 03/059516.

### BRIEF DESCRIPTION OF THE DRAWINGS

FIG. 1: Amount of produced PCR product according to Example 1. Herein, the amount is given in LightCycler units depending on the number of cycles.

FIG. 2: Analysis of the PCR setups of Example 1 on a 2% agarose gel. It can be seen that the amount of double-stranded DNA decreases and the amount of single-stranded DNA increases as the competitor proportion increases.

FIG. 3: Agarose gel, on which 5 μl-samples of reactions were analyzed parallel according to Example 2 with a competitor proportion of 50% and 87.5%, respectively. In each case, the reactions were stopped after the given number of cycles. It can be seen that less product is produced with a competitor proportion of 87.5%.

FIG. 4: Measurement of the intensity of the hybridization signals according to Example 2. The intensity was measured by averaging the signal strength (measured shade of gray) over the entire region of the spots. The gray value averaged over the spot-free region (background) was subtracted from this value. The signal intensities thus calculated were standardized (highest value=100%) and plotted against the number of cycles.

FIG. 5: Results according to Example 3. As expected, only one dominating product is detected in the gel analysis of the symmetrical reactions for all reactions, wherein an amplification took place. This is the double-stranded PCR product. Contrarily to this, 3 dominating products are detected in all detectable asymmetrical reactions. Two further bands corresponding to different structures of the labeled single strand are obtained beside the double-stranded template. Therefore, gel analysis shows that the asymmetrical amplification leads to a single strand surplus.

FIG. 6: Array layout according to Example 4. Black fields correspond to the probe C2938T, gray fields correspond to the probe C2938 WT.

FIG. 7: Detection of the hybridization signals according to Example 4. The hybridization results exhibit the following pattern: The probes for the mutation C2938T exhibit strong signals, while the wild type probes and the detection variants exhibit significantly lower signals. This is corresponding to the expectations. It is ostentatious that the signals are further enhanced by addition of the structure breakers. Therefore, a combination of competitor and secondary structure breaker is particularly useful for achieving good signal strengths.

FIG. 8: Detection of the hybridization signals according to Example 5. The hybridization results exhibited the following pattern: The probes for the mutation C2938T exhibited strong signals, while the wild type probes and the detection variants exhibit significantly lower signals. This is corresponding to the expectations. It is ostentatious that significantly higher signals are already achieved when adding one of the two structure breakers.

FIG. 9: Arrangement of the probes according to Example 6. Each of the 16 thick-lined squares contained 16 array elements equipped with the probes shown for the square in the lower left region of the illustration.

FIG. 10. Detection of the hybridization signals according to Example 6. FIG. 10a shows the fluorescence signals after hybridization of a PCR with the addition of structure breakers. The picture was made in the slide scanner with Laser-Power 70 and Photomultiplier 80. FIG. 10b shows the negative control, however, without addition of the corresponding structure breakers. The scanner settings were Laser-Power 100 and Photomultiplier 75. It is to be noted that FIG. 10b was thus recorded with a significantly higher laser and photomultiplier performance. Therefore, the signals on this chip only appear to be stronger.

FIG. 11. Depiction of the standardized measuring results from the hybridization of a PCR with addition and in absence of structure breakers according to Example 6. The ratio of hybridization signal with/without structure breaker (+O/−O) is plotted in addition.

FIG. 12: Scanner recordings of the hybridization results according to Example 7. The hybridized and washed chips were read out in a slide scanner (Scanarray4000, GSI Lumonics). Both arrays were recorded with identical scanner settings (Laser-Power 70 and Photomultiplier 80). FIG. 12a shows the fluorescence signals after hybridization of a PCR with the addition of structure breakers, whereas FIG. 12b shows the reaction without the addition of the corresponding structure breakers.

FIG. 13: Assignment of the individual probes to the array elements according to Example 8. Array elements occupied by the match probe are depicted in white, probe elements occupied by the deletion probe are depicted in black, and probe elements occupied by the insertion probe are depicted in gray.

FIG. 14: Hybridization signals according to Example 8 of the reaction stopped after 10 and 30 cycles, respectively.

FIG. 15: Setup of the array according to Example 9. Each of the 16 thick-lined squares contained 16 array elements equipped with the probes shown for the square in the lower left region of the illustration.

FIGS. 16a and 16b: Hybridization results according to Example 9.

FIG. 17: Evaluation of the recorded pictures according to Example 9 by means of the picture evaluation software Iconoclust® (Clondiag Chip Technologies). The hybridization intensity of the respective probes is shown for the respective reactions.

### DETAILED DESCRIPTION OF THE INVENTION

According to the present invention, nucleic acid molecules to be detected are referred to as ‘target’. Within the scope of the present invention, targets are therefore such nucleic acid molecules that are detected by means of a hybridization against probes arranged on a probe array. Within the scope of the present invention, these are DNA molecules, which were produced from a template by means of PCR amplification. Normally, these target molecules comprise sequences of 40 to 10,000 bases in length, preferably of 60 to 2,000 bases in length, also preferably of 60 to 1,000 bases in length, particularly preferably of 60 to 500 bases in length and most preferably of 60 to 150 bases in length. Their sequence comprises the sequence of the primers as well as the sequence regions of the template, which are defined by the primers. Generally, the target molecules can be single-stranded or double-stranded nucleic acid molecules, one or both strands whereof are labeled radioactively or non-radioactively, so that they can be detected by means of a detection method common in the prior art.

According to the present invention, a ‘target sequence’ is the sequence region of the target, which is detected by means of hybridization with the probe. According to the present invention, this is also referred to as this region being addressed by the probe.

According to the present invention, ‘probes’ are nucleic acid molecules of defined and known sequence, which are used for the detection of target molecules in hybridization methods. Typically, according to the present invention, probes are single-stranded nucleic acid molecules or molecules of nucleic acid analogs, preferably single-stranded DNA molecules or RNA molecules having at least one sequence region, which is complementary to a sequence region of the target molecules. With respect to detection method and application, the probes can be immobilized on a solid support substrate, for example in the form of a microarray. Moreover, depending on the detection method, they can be labeled radioactively or non-radioactively, so that they can be detected by means of detection reactions common in the prior art.

Usually, ‘array’ means the arrangement of molecules, e.g. probes, on a support. Herein, the arrangement of the molecules and probes, respectively, can be generated by means of covalent or non-covalent interactions. A position within the arrangement, i.e. within the array, is usually referred to as spot.

Conventional microarrays within the scope of the present invention comprise about 2 to about 80,000, preferably about 10 to about 65,000, particularly preferably about 20 to about 1,024 different species of probe molecules on an area of several mm² to several cm², preferably about 1 mm² to 10 cm², particularly preferably 2 mm² to 2 cm² and most preferably about 4 mm² to 1 cm². For example, a conventional microarray has 4 to 65,536 different species of probe molecules on an area of 2 mm×2 mm.

The support, which usually is also referred to as substrate or matrix, can be e.g. an object support or a wafer. The entirety of molecules applied in array-like arrangement and support is also often referred to as ‘chip’, ‘microarray’, ‘DNA chip’, ‘probe array’, etc.

A short DNA or RNA oligonucleotide (about 12 to 30 bases), which is complementary to a region of a larger DNA or RNA molecule and has a free 3-OH group at its 3′-end, is usually referred to as ‘primer’. Due to this free OH group, the primer can serve as substrate for arbitrary DNA or RNA polymerases, which synthesize nucleotides to the primer in the 5′-3′-direction. Herein, the sequence of the newly synthesized nucleotides is predetermined by the sequence of the template hybridized with the primer, which is located beyond the free 3′-OH group of the primer. Primers of conventional length comprise 12 to 50 nucleotides, preferably 15 to 30 nucleotides.

Usually, a double-stranded nucleic acid molecule or a nucleic acid strand serving as template for the synthesis of complementary nucleic acid strands is referred to as ‘template’ or ‘template strand’.

The formation of double-stranded nucleic acid molecules or duplex molecules from complementary single-stranded nucleic acid molecules is referred to as ‘hybridization’. Within the scope of a hybridization, e.g. DNA-DNA duplexes, DNA-RNA duplexes, or RNA-RNA duplexes can be produced. By means of a hybridization, duplexes with nucleic acid analogs can also be produced, like e.g. DNA-PNA duplexes, RNA-PNA duplexes, DNA-LNA duplexes, and RNA-LNA duplexes. Hybridization experiments are usually used to detect the sequence complementarity and with it the identity of two different nucleic acid molecules.

To this end, e.g. a single-stranded nucleic acid molecule, the probe, can be immobilized on a support and incubated with a variety of other single-stranded nucleic acid molecules, the targets. If sequence complementarity exists between the probe and one of the single-stranded nucleic acid molecules, the target molecule binds to the probe. If the target molecules are labeled, the hybridization event can be detected by means of applying the correspondent detection reaction, after the unbound target molecules have been removed by means of washing.

The person skilled in the art knows that the formation of stable double-stranded molecules depends on the stringency of the reaction conditions.

Stringent reaction conditions are such conditions, which allow the formation of a double-stranded nucleic acid molecule from single-stranded molecules only in case that a hundred percent or a high complementarity of the single strands is given.

According to the present invention, a high complementarity of single strands is given if the nucleic acid sequences of the single-stranded molecules have a complementarity of at least 60%, preferably of at least 70%, also preferably of at least 80%, particularly preferably of at least 90%, in particular preferably of at least 95%, and most preferably of at least 98%.

The person skilled in the art knows that the stringency of the hybridization depends on the temperatures prevailing during the hybridization and the washing steps and on the salt concentrations and pH values of the hybridization and washing buffers.

An increased stringency can be achieved by using increasingly more diluted salt solutions during hybridization. Increased stringency of the hybridization can also be achieved by adding destabilizing agents like e.g. formamide to the hybridization or washing buffer. Typical stringent conditions are e.g. given in Sambrook et al. (vide supra). Here, we can e.g. have 6×SSPE, 0.1% SDS at 50 to 65° C.

Reaction conditions, which only allow the formation of double-stranded nucleic acid molecules having only limited complementarity within the scope of a hybridization reaction, are referred to as low-stringency conditions. According to the present invention, nucleic acid molecules have a limited complementarity, if they have a complementarity of 20%, preferably of at most 30%, particularly preferably of at most 40%, in particular preferably of at most 50%, and most preferably of at most 60% regarding their sequence.

Two nucleic acid sequences are referred to as complementary, if the sequences can form hydrogen bonds between the nucleotides of the single nucleic acid molecules due to their base sequence, and, correspondingly, double-stranded nucleic acid molecules are formed. The case that a hydrogen bond can be formed between all of the opposite nucleotide pairs is referred to as 100% complementarity.

In the method according to the present invention, every molecule causing a preferred amplification of only one of the two template strands present in the PCR reaction can be employed as competitor in the PCR. According to the present invention, competitors can be proteins, peptides, DNA ligands, intercalators, nucleic acids, or analogs thereof.

According to the present invention, proteins and peptides, respectively, which are capable of binding single-stranded nucleic acids with sequence specificity and which have the above-defined properties, are preferably used as competitors. According to the present invention, in particular nucleic acid molecules and nucleic acid analog molecules are preferably employed as secondary structure breakers.

According to the present invention, the formation of one of the two template strands is substantially inhibited by initial addition of the competitor to the PCR during the amplification. According to the present invention, ‘substantially inhibited’ means that within the scope of the PCR a single strand surplus and an amount of the other template strand are produced, which suffice to allow an efficient detection of the amplified strand by means of the hybridization. Therefore, the amplification does not follow exponential kinetics of the form 2ⁿ (with n=number of cycles), but rather attenuated amplification kinetics of the form <2ⁿ.

According to the present invention, the single strand surplus of the amplified strand, which is achieved by the PCR method according to the present invention, in relation to the non-amplified strand has the factor 1.1 to 1,000, preferably the factor 1.1 to 300, also preferably the factor 1.1 to 100, particularly preferably the factor 1.5 to 100, also particularly preferably the factor 1.5 to 50, in particular preferably the factor 1.5 to 20 and most preferably the factor 2 to 10.

Typically, the function of a competitor will be to bind selectively to one of the two template strands and therefore to inhibit the amplification of the corresponding complementary strand. Therefore, competitors can be single-stranded DNA- or RNA-binding proteins having specificity for one of the two template strands to be amplified in a PCR. They can also be aptamers sequence-specifically binding only to certain regions of one of the two template strands to be amplified.

Nucleic acids or nucleic acid analogs are preferably employed as competitors in the method according to the present invention. Conventionally, the nucleic acids and nucleic acid analogs, respectively, will act as competitors of the PCR by either competing against one of the primers used for the PCR for the primer binding site or by being capable of hybridizing with a region of a template strand to be detected due to a sequence complementarity. According to the present invention, this region is not the sequence detected by the probe. According to the present invention, such nucleic acid competitors are enzymatically not extendable.

The nucleic acid analogs can be e.g. so-called peptide nucleic acids (PNA). However, nucleic acid analogs can also be nucleic acid molecules, in which the nucleotides are linked to one another via a phosphothioate bond instead of a phosphate bond. They can also be nucleic acid analogs, wherein the naturally occurring sugar components ribose and deoxyribose, respectively, have been replaced with alternative sugars like e.g. arabinose or trehalose (‘trehalose nucleic acid’, TNA) etc. Furthermore, the nucleic acid derivative can be ‘locked nucleic acid’ (LNA). Further conventional nucleic acid analogs are known to the person skilled in the art.

DNA or RNA molecules, in particular preferably DNA or RNA oligonucleotides and their analogs, respectively, are preferably used as competitors.

Depending on the sequence of the nucleic acid molecules and nucleic acid analogs, respectively, employed as competitors, the inhibition of the amplification of one of the two template strands within the scope of the PCR reaction is based on different mechanisms. By the example of a DNA molecule, this is discussed in the following.

If e.g. a DNA molecule is used as competitor, it can have a sequence, which is at least partially identical to the sequence of one of the primers used for the PCR in such a way that a specific hybridization of the DNA competitor molecule with the corresponding template strand is possible under stringent conditions. As, according to the present invention, the DNA molecule used for competition in this case is not extendable by a DNA polymerase, the DNA molecule competes for binding to the template against the respective primer during the PCR reaction. According to the ratio of the DNA competitor molecule and the primer, the amplification of the template strand defined by the primer can thus be inhibited in such a way as to significantly reduce the production of this template strand. Herein the PCR proceeds according to exponential kinetics higher than would be expected with respect to the amounts of competitors used. This way, a single strand excess emerges in an amount that is sufficient for the efficient detection of the amplified target molecules by means of hybridization.

According to the present invention, the nucleic acid molecules and nucleic acid analogs, respectively, used for competition must not be enzymatically extendable in this preferred embodiment.

‘Enzymatically not extendable’ means that the DNA or RNA polymerase used for the amplification cannot use the nucleic acid competitor as primer, i.e. it is not capable of synthesizing the corresponding opposite strand of the template 3′ from the sequence defined by the competitor.

Alternatively to the depicted possibility, the DNA competitor molecule can also have a sequence complementary to a region of the template strand to be detected not being addressed by one of the primer sequences and which is enzymatically not extendable. Within the scope of the PCR, the DNA competitor molecule will then hybridize to this template strand and correspondingly block the amplification of this strand.

The person skilled in the art knows that the sequences of DNA competitor molecules or generally nucleic acid competitor molecules can be selected correspondingly. If the nucleic acid competitor molecules have a sequence, which is not identical to the sequence of one of the primers used for the PCR, but is complementary to another region of the template strand to be detected, this sequence, according to the present invention, is to be selected in such a way that it does not fall within the region of the template sequence, which is detected with a probe within the scope of the hybridization. This is necessary due to the fact that, in the method according to the present invention, there does not have to occur a processing reaction between the PCR and the hybridization reaction. If a nucleic acid molecule, which falls within the region to be detected, were used as competitor, it would compete for binding to the probe against the single-stranded target molecule.

Such competitors preferably hybridize near the template sequence, which is detected by the probe. Here, according to the present invention, the position specification ‘near’ is to be understood in the same way as given for secondary structure breakers (see below). However, the competitors according to the present invention can also hybridize in the immediate proximity of the sequence to be detected, i.e. in exactly one nucleotide's distance from the target sequence to be detected.

If enzymatically not extendable nucleic acids and nucleic acid analogs, respectively, are used as competing molecules, they are to be selected according to their sequence and structure in such a way that they cannot be enzymatically extended by DNA or RNA polymerases. Preferably, the 3′-end of a nucleic acid competitor is designed in such a way that it has no complementarity to the template and/or has at its 3′-end another substituent instead of the 3-OH group.

If the 3′-end of the nucleic acid competitor has no complementarity to the template, regardless of whether the nucleic acid competitor binds to one of the primer binding sites of the template or to one of the sequences of the template to be amplified by means of the PCR, the nucleic acid competitor cannot be extended by the conventional DNA polymerases due to the lack of base complementarity at its 3′-end. This type of non-extensibility of nucleic acid competitors by DNA polymerases is known to the person skilled in the art. Preferably, the nucleic acid competitor has no complementarity to its target sequence at its 3′-end with respect to the last 4 bases, particularly preferably to the last 3 bases, in particular preferably to the last 2 bases and most preferably to the last base. Such competitors can also have non-natural bases, which do not allow hybridization, in the mentioned positions.

According to the present invention, nucleic acid competitors, which are enzymatically not extendable, can also have a 100% complementarity to their target sequence, if they are modified in their backbone or at their 3′-end in such a way that they are enzymatically not extendable.

If the nucleic acid competitor has at its 3′-end a group other than the OH group, these substituents preferably are a phosphate group, a hydrogen atom (dideoxynucleotide), a biotin group, or an amino group. These groups cannot be extended by the conventional polymerases.

The use of a DNA molecule, which competes for binding to the template against one of the two primers used for the PCR and which was provided with an amino link at its 3′-end during chemical synthesis, as a competitor in a method according to the present invention is particularly preferred. Such competitors may have a 100% sequence complementarity to their target sequence.

However, nucleic acid competitors, like e.g. PNAs, according to the present invention do not have to have a blocked 3′-OH group or a non-complementary base at their 3′-end, as they are not recognized by the DNA polymerases because of the backbone modified by the peptide bond and thus are not extended. Other corresponding modifications of the phosphate group, which are not recognized by the DNA polymerases, are known to the person skilled in the art. Belonging thereto are, inter alia, nucleic acids with backbone modifications like e.g. 2′-5′ amide bonds (Chan et al. (1999) J. Chem. Soc., Perkin Trans. 1, 315-320), sulfide bonds (Kawai et al. (1993) Nucleic Acids Res., 1 (6), 1473-1479), LNA (Sorensen et al. (2002) J. Am. Chem. Soc., 124 (10), 2164-2176) and TNA (Schoning et al. (2000) Science, 290 (5495), 1347-1351).

According to the present invention, several competitors hybridizing to different regions of the template (e.g. inter alia the primer binding site) can also simultaneously be employed in a PCR. The efficiency of the hybridization can additionally be increased, if the competitors have properties of secondary structure breakers.

In an alternative embodiment, the DNA competitor molecule can also have a sequence complementary to one of the primers. Depending on the ratio of antisense DNA competitor molecule and primer, such e.g. antisense DNA competitor molecules can then be used to titrate the primer in the PCR reaction, so that it does no longer hybridize with the corresponding template strand and, correspondingly, only the template strand defined by the other primer is amplified. The person skilled in the art is aware of the fact that, in this embodiment of the invention, the nucleic acid competitor can, but does not have to, be enzymatically extendable.

If, according to the present invention, nucleic acid competitors are mentioned, this includes nucleic acid analog competitors, unless a different meaning arises from the respective context. The nucleic acid competitor can bind to the corresponding strand of the template reversibly or irreversibly. The bond can take place via covalent and/or non-covalent interactions.

Preferably, the bond of the nucleic acid competitor takes place via non-covalent interactions and is reversible. Particularly preferred, the bond to the template takes place via formation of Watson-Crick base pairings.

The sequences of the nucleic acid competitors normally adapt to the sequence of the template strand to be detected. In the case of antisense primers, however, they adapt to the primer sequences to be titrated, which are in turn defined by the template sequences, though.

PCR amplification of nucleic acids is a standard laboratory method, with whose various possibilities of variation and development the person skilled in the art is familiar. In principle, a PCR is characterized in that the double-stranded nucleic acid template, usually a double-stranded DNA molecule, is first subjected to heat denaturation for 5 minutes at 95° C., whereby the two strands are separated from each other. After cooling down to the so-called ‘annealing temperature’ (defined by the primer with the lower melting temperature), the forward and reverse primers present in the reaction solution accumulate at those sites in the respective template strands, which are complementary to their own sequences. The annealing temperature of the primers herein adapts to the length and base structure of the primers. It can be calculated on the basis of theoretical considerations. Information on the calculation of annealing temperatures can be found e.g. in Sambrook et al. (vide supra).

Annealing of the primers, which typically is performed within a range of temperature between 40 to 75° C., preferably between 45 to 72° C. and in particular preferably between 50 to 72° C., is followed by an elongation step, in which deoxyribonucleotides are linked with the 3′-end of the primers by the activity of the DNA polymerase present in the reaction solution. Herein, the identity of the inserted dNTPs depends on the sequence of the template strand hybridized with the primer. As normally thermostable DNA polymerases are employed, the elongation step usually runs at 68 to 72° C.

In a symmetrical PCR, an exponential increase of the nucleic acid region of the target defined by the primer sequences is achieved by repeating this described cycle of denaturation, annealing of the primers and elongation of the primers.

With respect to the buffer conditions of the PCR, the usable DNA polymerases, the production of double-stranded DNA templates, the design of primers, the selection of the annealing temperature, and variations of the classic PCR, the person skilled in the art has numerous works of literature at his disposal. Therefore, within the scope of the present invention, a PCR is not only understood to be a classic PCR, but also a PCR variation like RT-PCR, inverse PCR, quantitative PCR, multiplex PCR, hot-start PCR, touchdown PCR and the like (also see Sambrook et al., Molecular Cloning—A Laboratory Manual, Band 2, 3. Auflage, Cold Spring Harbor Laboratory Press, Cold Spring Harbor, N.Y., 2001)

It is familiar to the person skilled in the art that also e.g. single-stranded RNA, like e.g. mRNA, can be employed as template. Usually, it is previously transcribed into a double-stranded cDNA by means of a reverse transcription.

In a preferred embodiment of the method, a thermostable DNA-dependent polymerase is used as polymerase. In a particularly preferred embodiment, a thermostable DNA-dependent DNA polymerase is used, which is selected from the group consisting of Taq-DNA polymerase (Eppendorf, Hamburg, Germany and Qiagen, Hilden, Germany), Pfu-DNA polymerase (Stratagene, La Jolla, USA), Tth-DNA polymerase (Biozym Epicenter Technol., Madison, USA), Vent-DNA polymerase, DeepVent-DNA polymerase (New England Biolabs, Beverly, USA), Expand-DNA polymerase (Roche, Mannheim, Germany).

The use of polymerases, which have been optimized from naturally occurring polymerases by means of specific or evolutive modification, is also preferred. When performing the method according to the present invention, the use of the Taq-polymerase by Eppendorf (Germany) and of the Advantage cDNA Polymerase Mix by Clontech (Palo Alto, Calif., USA) is in particular preferred.

Reaction buffers for thermostable DNA-dependent DNA polymerases, as they are commercially available, are especially used as reaction buffers within the scope of the method according to the present invention. In particular, the reaction buffer delivered together with the respectively used polymerase is used here. Particularly preferred is the use of the cDNA PCR reaction buffer by Clontech (Palo Alto, Calif., USA).

In particularly preferred embodiments of the method according to the present invention, the ionic strength of the PCR reaction buffer can be increased by the addition of salts. This can be done by means of adding monovalent ions like K⁺, Na⁺ or other ions. Particularly preferably, the cDNA PCR reaction buffer by Clontech is used in connection with an increased potassium acetate concentration. Usually, the concentration of the monovalent metal ions lies between 0 mM and 2 M, preferably between 20 mM and 200 mM and particularly preferably between 40 mM and 120 mM.

Though the increased ionic strength does not influence the PCR reaction itself, it is advantageous for the efficient hybridization of the PCR fragments to the probes.

In a preferred embodiment, the primer and competitor competing for a binding site preferably have the same binding properties (Tm value, annealing temperature or melting temperature). Alternatively, competitor and primer can also differ in their binding properties. In a preferred case, the competitor has a lower melting temperature than the competing primer. This is particularly preferred in case the PCR shall be performed in a process that is continuous but has two steps concerning temperature regime and amplification kinetics.

In this case, during the first phase of the amplification in the annealing step of the PCR a temperature is selected, at which the primer, but not the competitor, binds to the template strand. Under these conditions, the amplification takes place symmetrically. There is no single strand surplus. In the second amplification phase, a single strand surplus is produced by performing the hybridization step of the PCR cycle at a, preferably lowered, temperature, at which both primer and competitor bind. This two-step temperature regime is analogously also preferred with the use of competitors binding to primers as well as with the employment of structure breakers (see above).

In a special embodiment, the primer and competitor component can be located on the same molecule (primer-competitor). In this embodiment, the primer-competitor has a hairpin structure, in which the 3′-region is equivalent to the primer sequence, while the 5′-region has a sequence that is at least partially complementary to the primer. In this embodiment, the hybridization of competitor module and primer competes against the hybridization of the primer to the template. Thus, this is the already mentioned antisense-competitor setup. In a preferred embodiment, primer module and competitor module of the primer-competitor are linked with each other by a region, which cannot serve as a template for polymerases. Particularly preferred, these are base-free regions or regions with non-natural bases, which cannot undergo base pairing with the natural bases. Also particularly preferred in this connection is the substitution of the bases by other components or the connection of primer and competitor module by means of spacer structures, respectively, which do not represent a DNA, e.g. polyethylene glycol derivatives, peptides, terpenes, and others. These regions prevent the polymerase from binding the 3′-end of the template strand complementary to the primer-competitor after hybridization of the primer-competitor and from copying the competitor region. This would lead to the formation of a hairpin structure at the end of the extended template, which could then in turn be extended by the polymerase in dependence of the same strand (‘self priming’).

In a particularly preferred embodiment of the antisense-competitor setup, the stability (Tm value) of the hybrid of primer and competitor component is lower than the stability of the hybrid of primer and template. In this case, the PCR is performed in a process that is continuous but has two steps concerning the temperature regime and amplification kinetics, as described above.

In this case, the formation of the primer-competitor hybrid is prevented in the first, the PCR phase, by selecting an annealing temperature higher than the Tm value of the primer-competitor hybrid. In the second phase, the hybridization temperature is lowered, so that the hybridization of primer and template is partially or completely prevented by formation of the primer-competitor hybrid.

Particularly preferably, the amplification is performed as a one-phase process. In this process, particularly preferably, primer and competitor are discrete molecules, wherein the competitor is enzymatically not extendable and competes against the primer for binding to the template.

The ratio of primer and competitor with respect to the molar amounts is between 0.01 and 0.99, preferably between 0.05 and 0.8 particularly preferably between 0.1 and 0.5 at the beginning of the reaction.

Thus, according to the present invention, the competitor is present at least in equimolar amounts and preferably in 2-, 5-, 10-, 20-, 50-, 100-, 1,000-fold molar excess in relation to one of the PCR primers at the beginning of the reaction.

By means of the method according to the present invention, the hybridization signal can be increased by the factor 2 to 10,000, preferably by the factor 2 to 1,000, also preferably by the factor 2 to 500, particularly preferably by the factor 2 to 200, also particularly preferably by the factor 2 to 100, in particular preferably by the factor 2 to 60 and most preferably by the factor 2 to 30 in comparison with methods, wherein the target is amplified in a symmetrical PCR.

As already mentioned, a particularly preferred embodiment of the invention is a method, wherein at first a PCR is performed, whereto a nucleic acid competitor competing for binding to the corresponding target strand against one of the two primers used for the PCR, which is enzymatically not extendable, has been added at the beginning. Thereby, a single strand surplus of the target molecule emerges within the scope of the PCR, which can then be detected in the subsequent hybridization against a corresponding probe. Such a method can be performed e.g. in a microarray-based evaluation and has various advantages.

The particularly preferred method according to the present invention herein proceeds according to the following embodiment: First, a reaction mixture is prepared consisting of a template DNA and reagents allowing the PCR amplification of a region of the template DNA. Typically, these reagents are a reaction buffer, a thermostable DNA-dependent DNA polymerase, deoxynucleotide triphosphates (dNTP) and, where required, further components. One or more primer pairs having complementarity to the regions on each strand including the region to be amplified on the template, and a nucleic acid competitor, competing for the binding site on the template against one primer of the primer pair, which cannot serve as a primer because it is not enzymatically extendable itself, are also added to the reaction. According to the present invention, several primer pairs and competitors can also be employed simultaneously, so that several template regions are simultaneously amplified by a PCR in a single strand surplus. According to the present invention, several templates can also be employed.

The reaction mixture is, in suitable form, brought in contact with a microarray bearing probes, which are at least partially complementary to the strand of the amplification product to be expected. The reaction mixture provided with the probe array is first incubated under conditions allowing a PCR amplification. Subsequently, incubation is performed under conditions, which allow a hybridization of the amplification product with the probes of the microarray. As the primers used for the amplification have been correspondingly labeled previously and the identity and localization of the probes on the array are known, the hybridization pattern can be read out on the probe array by means of a suitable detection means and it can deliver information on which target molecules are contained in a sample. If necessary, the microarray and the reaction mixture are separated before the evaluation and the array is washed in a suitable buffer.

According to the present invention, the hybridization preferably takes place in the same buffer, in which the PCR reaction was performed. This buffer usually is a standard PCR buffer; preferably, the PCR buffer is increased in ionic strength by addition of ions (see above). Particularly preferably, the hybridization is performed in the cDNA reaction buffer by Clontech (Palo Alto, Calif., USA), whose ionic strength has been increased, preferably by addition of potassium acetate, to a final concentration of preferably 15 to 100 mM and particularly preferably of 50 to 90 mM.

The optimum hybridization temperature depends, inter alia, on the composition of the used buffer and on the length of the hybridized regions and lies between 0° C. and 65° C. With the use of the particularly preferred buffer system and a length of the hybridizing regions of 18 to 25 bases, the preferred hybridization temperature lies between 20° C. and 55° C., particularly preferably between 30° C. and 45° C. and most preferably between 35° C. and 45° C.

The experiments depicted further below show that the preferred embodiment of the method according to the present invention has several advantages. In the experiments depicted further below, DNA molecules having a defined sequence and a 3′-end, which is complementary to the target sequence of the template and can be extended by DNA polymerases, were used as primers, while the competitor was a DNA molecule of the same length and sequence in relation to a primer, wherein the 3′-end was blocked by means of an amino modification. Maintaining the same final concentration of the primer-competitor mixture, the ratio of primer and competitor was varied between 0% and 100% competitor proportion.

In this preferred embodiment, primer and competitor compete for the same binding site on the target sequence during the reaction. Depending on the selected primer-competitor ratio, a certain proportion of a template strand is bound by the competitor during the annealing step of the PCR. This proportion cannot be extended in the subsequent elongation step, so that the single strand bound by the competitor is still present as a surplus single strand after the elongation step. Contrarily to this, the opposite strand is completely transcribed, as no competitor has been added for it. The desired effect concerning the hybridization properties of the amplification product is that a single strand surplus exists during the entire course of the reaction, i.e. an asymmetrical amplification is performed.

However, when quantifying the amplification products during the entire course of the reactions, it can be observed that the amplification rate was reduced by addition of the competitor. However, the amplification rate was surprisingly reduced significantly less than would have been expected on the basis of calculations based on the selected primer-competitor ratio.

In addition, it was observed in hybridization experiments, wherein the same volumina as in the different PCR setups were used for hybridization, that in the case of an asymmetrical amplification the hybridization signals were increased by the factor 2 to 100 compared to the symmetrical amplification during the entire detectable course of the reaction, in particular with a competitor proportion of 50 to 90%, i.e. with an up to 9-fold molar surplus. This was surprising, as given such a high competitor proportion it was expected that the amplification is slowed down to such an extent, that the amount of amplification product produced is below the detection limit of the hybridization.

The PCR products produced according to the above-described principle of asymmetrical amplification therefore have a single strand surplus during the entire course of the reaction. Therefore, they can be detected much more sensitively in hybridization assays than could completely double-stranded DNA molecules produced by means of symmetrical amplification.

Further, it was observed that the PCR products produced by asymmetrical PCR according to the present invention do not only hybridize efficiently with probes on probe arrays in classic hybridization buffers, but that an efficient and specific hybridization can also take place in buffers, which are suitable as PCR buffers. This offers the possibility of performing detection reactions, wherein a PCR amplification is followed by a hybridization reaction, in one and the same buffer in a continuous process. In such a method, which is preferred according to the present invention, at the beginning of the detection reaction all components needed for the PCR amplification are combined, the reaction mixture is provided with an array and the reaction is subsequently subjected to a PCR temperature regime suitable for the amplification of the respective template and to a subsequent hybridization step.

The PCR amplification can also be performed as multiplex reaction. Here, preferably one primer pair together with a corresponding competitor is added for each of the templates to be amplified in the multiplex reaction. This is particularly preferred, if the multiplex reaction shall be performed in closed reaction chambers, where an addition of further components is not possible during the reaction.

Alternatively, the multiplex reaction can also be performed in a two-step process as it is described in Favis et al. (Nat. Biotechnol. (2000) 18(5):561-4). In this case, the multiplex reaction is performed by first adding a limited amount of a primer pair, whose 3′-end exhibits homology to the respective template, whose 5′-ends, however, are identical for all forward and reverse primers (universal region), for each fragment to be amplified. The second phase of the reaction is performed with a primer pair complementary to the universal regions. If such a multiplex method is applied, the use of only one competitor is particularly preferred. This competitor is of such a nature, that it competes for the binding site on the templates against one of the universal primers. It is also particularly preferred that one of the universal primers is designed as primer-competitor and the reaction is performed as described above for the use of a primer-competitor in the second phase.

It is a special embodiment for asymmetric multiplex reactions that the specific primers used in the first phase have a higher melting temperature than the universal primers used in the second phase of the reaction and the competitor. Under this condition, all components necessary for the two-step multiplex PCR and the asymmetrical amplification can be added at the beginning of the reaction. In this case, the two-step character of the reaction is achieved by means of a corresponding temperature regime. High annealing temperatures during the first phase guarantee a symmetrical amplification using the specific primers; lowered annealing temperatures during the second phase lead to the use of the universal primers and to asymmetrical amplification. This embodiment is particularly preferred, if the two-step form of the multiplex PCR shall be performed in a closed reaction chamber, so that an addition of further components is not possible during the reaction.

In a further embodiment, the asymmetrical multiplex PCR is performed in a continuous process of three phases concerning the amplification reaction. In this case, specific primer, universal primer, and competitor have a melting temperature decreasing in order of listing. The transition from specific to symmetrical universal and to asymmetrical amplification is achieved in each case by lowering the annealing temperature in the PCR program.

For the detection of the hybridization signals on the probe array, in most cases a labeling of the target is necessary, i.e. the target has to be equipped with a colorant, an anchor group or an otherwise detectable unit.

Within the scope of the present invention, the labeling of the target (the PCR products) is preferably achieved by the insertion of labeled primers. Alternatively, labeled monomers can also be used, however.

Particularly preferably, the labeling is achieved by adding the primer, which does not compete against the competitor, to the reaction as a labeled component.

Labeling is preferably based on the use of fluorophor-labeled primers and/or monomers. In connection with fluorescence detection, the method allows the labeling with arbitrary colorants, which can be coupled to nucleotide triphosphates or to oligonucleotides during or after the synthesis, for example Cy-colorants like Cy3 or Cy5 (Amersham Pharmacia Biotech, Uppsala, Sweden), Alexa colorants like Texas Red, Fluorescein, Rhodamine (Molecular Probes, Eugene, Oreg., USA) or lanthanides like samarium, ytterbium and europium (EG&G Wallac, Freiburg, Germany). Fluorescence-labeled primers are preferably used. Particularly preferably, labeling is done with Cy-colorants.

In an alternative embodiment of the method according to the present invention, the detection is based on the use of primers and monomers, respectively, which are coupled with anchor groups. Preferably used anchor groups are biotin, dioxigenin, and the like. The anchor groups are converted in a subsequent reaction with specifically binding components, for example Streptavidin conjugates or antibody conjugates, which are detectable themselves or trigger a detectable reaction. When anchor groups are employed, the conversion of the anchor groups into detectable units takes place subsequently to the hybridization.

In a further alternative embodiment of the method according to the present invention, detection methods are used, which yield as a result an adduct having a certain solubility product leading to a precipitation. I.e., substrates, which can be converted into a not easily soluble, usually colored product, are used for labeling. For example, enzymes catalyzing the conversion of a substrate into a not easily soluble product can be used in this labeling reaction. Examples for such reactions are the conversion of chromogenic substrates like tetramethylbenzidine (TMB)-containing, precipitating reagents by peroxidases (e.g. horseradish peroxidase, HRP) and the conversion of BCIP/NBT mixtures by phosphatases, respectively. Examples for highly sensitive detection methods, wherein a not easily soluble product is formed, are the Tyramide Signal Amplification System (TSA) and the Enzyme-Labeled Fluorescence (ELF) signal amplification (see R. Haugland, Molecular Probes, Handbook of Fluorescent Probes and Research Products, 9. Auflage, 152-166).

In a further alternative embodiment of the method according to the present invention, detection globules, in particular made of gold, are used as markers. The signal can preferably be enhanced by means of silver precipitation at the detection globules. In this manner, the above-mentioned detection method can be performed by means of not easily soluble products based on metal labelings. Herein, for example colloidal gold or defined gold clusters are coupled directly to the primer, which is not competing against the competitor, or are bound via certain mediator molecules like streptavidin or antibodies to the primer, which then must have a corresponding anchor group for these mediator molecules. It is also possible to insert the gold label by means of sandwich hybridization with the target. In all cases, an enhancement of the labeling by means of reaction with less precious metals, like e.g. silver, subsequently takes place. The detection of the regions enhanced by means of the specific reaction is performed by means of a very simple optical installation in transmitted light (contrast by shadowing effects) and/or incident light (contrast by reflection).

A further alternative embodiment concerning detection is based on the use of radioactively labeled structures or components like ³²P or ³H.

In certain embodiments, the labeling of the target can be entirely omitted, if the reading of the hybridization signals is based on a label-free detection method like for example mass spectroscopy or surface plasmon resonance.

In a further embodiment, the method is performed with a probe array described in the German Patent Application 101 42 643.7-44. In this case, the labeling of the target can also be omitted.

The performance of the preferred method according to the present invention, wherein the nucleic acid to be detected is first amplified by means of a PCR, wherein the amplification of one of the two template strands is inhibited by initial addition of a competitor, and the detection of the amplified single-stranded nucleic acid is performed by means of hybridization against a probe having a correspondingly complementary sequence, has various advantages. As the strand to be detected is produced in a surplus in the PCR reaction, the strand is automatically present in single-stranded form in the reaction mixture used for the hybridization. Thereby, the competition of the hybridization with the complementary strand in the solution against the hybridization with the complementary probe on the microarray is substantially eliminated. This competition appears with the use of double-stranded DNA as hybridization target, as it is produced by means of a PCR under usual conditions not according to the present invention. The efficiency of the hybridization with the probe on the microarray is increased in the method according to the present invention.

It is a further advantage of the method according to the present invention that the single strand surplus is present during the entire course of the amplification, without the necessity of the experimenter's intervention in the reaction system for producing this single strand surplus by means of addition of components at an advanced phase of the amplification. This intervention of the experimenter is, as mentioned above, characteristic for the classic linear or asymmetrical PCR. As such an intervention is not required in the method according to the present invention, the method has the advantage of being performable very easily and also in closed reaction containers, which are not accessible from the outside anymore after the start of the reaction.

It is a further advantage of the method according to the present invention that the amplification of the target sequences and the hybridization of the amplification products can be performed in one and the same buffer system without adding further components or changing the washing buffer and/or hybridization buffers. Thereby, for example in microarray-based detection methods, the entire assay can be performed without the necessity of the experimenter's intervention during the reaction.

It is another advantage of the method according to the present invention that despite the presence of the competitor the single-stranded target molecules are amplified in amounts sufficient for the hybridization.

One aspect of the present invention is also a method for the efficient detection of nucleic acids by means of hybridization, wherein the hybridization of the probe and the target, which respectively have complementary sequence regions, is performed in the presence of a molecule capable of binding to the target near the sequence region of the target hybridizing with the probe, wherein a pre-incubation of the target with the molecule is not necessary.

It has now surprisingly been found that the efficiency of the hybridization between probe and target can be substantially increased and therefore the detection be improved by means of initially adding molecules to hybridization assays, wherein a single-stranded nucleic acid probe binds to a single-stranded nucleic acid target, wherein both the probe and the target have complementary sequence regions and the molecule binds to the target near the sequence region of the target hybridizing with the probe.

Herein, a pre-incubation of the target with said molecule before the actual hybridization is surprisingly not necessary. Therefore, an intervention of the experimenter during the hybridization process is not required.

Without intending to be bound by a scientific theory, it is assumed that this increase in efficiency of the hybridization is due to the fact that the bond of the molecule to a target region located near the sequence region, which is detected by means of hybridization with the probe, prevents the formation of secondary structures in the target sequence.

According to the present invention, such molecules can be proteins, peptides, nucleic acids or analogs thereof, intercalators and other chemical compounds, which specifically bind near the target region, which is detected by means of hybridization with the probe. In the following, the molecules having the effect according to the present invention are also referred to as secondary structure breakers.

According to the present invention, proteins and/or peptides, which are capable of binding single-stranded nucleic acids with sequence specificity and which have the above-defined properties, are preferably used as secondary structure breakers. According to the present invention, the use of nucleic acid molecules and nucleic acid analog molecules as secondary structure breakers is particularly preferred.

The nucleic acid analogs can be e.g. the already mentioned PNAs, phosphothioate-modified nucleic acids or nucleic acids otherwise modified regarding their bases or their sugar backbone. These have already been specified above in context with the PCR competitors. Generally, said nucleic acid analogs must be characterized in that they are able to specifically bind to the target near the target region, which is detected by means of hybridization with the probe.

In a particularly preferred embodiment of the invention, nucleic acid molecules being DNA or RNA molecules are used as secondary structure breakers. According to the present invention, such nucleic acid secondary structure breakers must have sequences allowing them to specifically hybridize near the target region, which is detected by means of hybridization with the probe.

If the secondary structure breakers according to the present invention are nucleic acids or nucleic acid analogs, these comprise, according to the present invention, between 5 and 100 bases, preferably between 10 and 50 bases, particularly preferably between 10 and 30 bases and most preferably between 15 and 25 bases.

Within the scope of the present invention it has surprisingly been found that, if the secondary structure breakers according to the present invention are nucleic acids and/or nucleic acid analogs, these do not have to hybridize with the target in immediate proximity to the probe, i.e. that there must neither be a distance nor an overlapping, between the hybridized secondary structure breaker and the probe, as it is requested in the prior art, but they only have to bind to the target near the region of the target sequence, which is detected by means of hybridization with the probe.

According to the present invention, ‘near’ means that if nucleic acid and/or analogs thereof are used as secondary structure breakers, there can be a distance of at least 20, preferably at least 15, also preferably at least 10, particularly preferably at least 8, also particularly preferably at least 5, in particular preferably at least 4, also in particular preferably at least 3 and most preferably at least 2 nucleotides or 1 nucleotide between the secondary structure breaker hybridized with the target and the probe hybridized with the target.

If proteins, peptides, aptamers etc, or nucleic acid molecules and/or molecules of nucleic acid analogs, which are enzymatically not extendable, are used as secondary structure breakers, these can also bind to the target molecule in immediate proximity of the target sequence, which is detected by the probe, i.e. without a distance from it and without an overlapping.

If nucleic acids and/or analogs thereof are used as secondary structure breakers according to the present invention, these can, but do not have to, have a sequence or be chemically modified in such a way that they are enzymatically not extendable e.g. in a PCR reaction.

The question whether they have a non-enzymatic extendable sequence or are correspondingly chemically modified depends on whether they are used not until for hybridization or, as in the method depicted above, are added to a PCR reaction already at the beginning of the amplification of the target.

If they are already added to the PCR reaction at the beginning, the secondary structure breakers must have a sequence or must be chemically modified in such a way that they cannot be enzymatically extended by a DNA or RNA polymerase. Here, the requirements regarding the sequence or the chemical modification, respectively, of the secondary structure breaker nucleic acid molecules have already been depicted above in context with the nucleic acid competitors and can be transferred to the secondary structure breakers, if they are nucleic acids or analogs thereof. However, the person skilled in the art is aware of the fact that secondary structure breakers can, regarding their length, be larger than the competitors used for the asymmetrical PCR according to the present invention. If enzymatically non-extendable secondary structure breakers are already added to a PCR at the beginning, a competitor according to the present invention, which inhibits the amplification of one of the two template strands, does not additionally have to be added to the PCR reaction. In this case, an increase of the hybridization signals is already achieved by the addition of the secondary structure breaker to the PCR reaction, i.e. in a preferred embodiment of the invention, the secondary structure breaker can also be added to a symmetrical PCR in order to increase hybridization signals.

According to the present invention, the nucleic acid secondary structure breakers can be added to the PCR reaction in addition to the nucleic acid competitors, which e.g., as mentioned above, can compete against one of the primers for binding to the template. According to the present invention, this embodiment is particularly preferred, as in this way a single strand surplus is achieved in the PCR and the efficiency of the hybridization between probe and target is increased by the presence of the secondary structure breaker in the subsequent hybridization without the experimenter having to intervene during the course of the entire process. The advantages in comparison with the prior art, which were already depicted in the above, apply to this embodiment of the method to full extent.

However, the secondary structure breaker nucleic acids and/or analogs thereof can also be added to the PCR reaction exclusively. Then, on the one hand, they function as nucleic acid competitors during the PCR amplification and cause an amplification of only one template strand in surplus, on the other hand they act as secondary structure breakers during the hybridization and correspondingly increase the efficiency of the hybridization. In this likewise preferred embodiment of the invention, the experimenter does again not have to intervene. As PCR competitor and hybridization secondary structure breaker are identical in this case, the method can be performed very cost-efficiently.

As mentioned, one and the same molecule acts as competitor and as structure breaker in preferred embodiments of the invention. In particular, this is the case if the template is so short that the target sequence and the sequence addressed by the primer are located in indirect or immediate proximity or if the asymmetric amplification is achieved by binding of the competitor to a region of the template, which is different from the primer binding site.

If the secondary structure breakers are e.g. thermostable proteins, peptides, aptamers, or other ligands having the above-mentioned specificity regarding binding to the target, these can of course also be employed at the beginning of the PCR amplification in the detection method. This embodiment of the method according to the present invention then again has the mentioned advantages.

If nucleic acids and/or nucleic acid analogs acting as secondary structure breakers are added to the reaction mixture after the PCR amplification, i.e. only for the hybridization, these do not have to have a sequence or be chemically modified in such a way that they are not enzymatically extendable. Though an additional working step is necessary in this embodiment according to the present invention, viz the addition of the secondary structure breakers to the reaction solution, this embodiment of the invention is still advantageous as hereby the efficiency of the hybridization of the single-stranded target produced in the PCR to the probe can be significantly increased because no secondary structures form in the target.

The secondary structure breakers according to the present invention have in common that they can be added together with targets and probes at the beginning of the hybridization. A general pre-incubation of the secondary structure breakers with the target, i.e. for example a pre-annealing of the corresponding secondary structure breaker DNA oligonucleotides with the target DNA molecules, is not necessary according to the present invention.

In experiments, however, it has surprisingly been found that secondary structure breakers being oligonucleotides or nucleic acids, respectively, also cause an additional increase of the hybridization signal of problematic targets in a continuous PCR hybridization assay, as described above, wherein nucleic acids competing against one of the primers for binding to the template were additionally used as competitors, if they are added to the reaction mixture at the beginning of the reaction. In this case, the secondary structure breakers must not be enzymatically extendable.

The structure breakers are preferably nucleic acid molecules or nucleic acid analogs particularly preferably DNA or PNA molecules.

In a particularly preferred embodiment, the structure breakers are added to the reaction mixture before the PCR reaction. If the structure breakers are RNA or DNA molecules, their structure or chemical composition is designed in such a way that they cannot be extended by the polymerase by means of DNA synthesis. Preferably, the 3′-end of a nucleic acid structure breaker is designed in such a way that it has no complementarity to the template and/or has a different substituent at its 3′-end instead of the 3-OH group. This different substituent preferably is a phosphate group, a hydrogen atom (so that a dideoxynucleotide emerges), biotin, or an amino group, the latter optionally linked with a linker component common in chemical DNA synthesis. Concerning the complementarity of the 3′-end, it is referred to the nucleic acid competitors, as the requirements are identical. In a preferred embodiment, secondary structure breakers are enzymatically not extendable and can have a 100% complementarity to their target sequence.

It is also preferred that the structure breakers are added to the reaction mixture only after completed amplification, immediately before the hybridization step This embodiment is particularly preferred, if the reaction is not performed within a closed reaction chamber, where an addition of further components during the course of the reaction would not be possible.

The optimal molar ratios of structure breaker and target at the end of the reaction normally lie between 1 to 100,000, preferably between 2 to 1,000, particularly preferably between 5 to 500 and most preferably between 5 to 100.

The person skilled in the art is aware of the fact that the different aspects of the invention can also be combined with each other in order to achieve an efficient amplification of single-stranded nucleic acid molecules and to detect these efficiently by means of hybridization in a continuous process.

In a further embodiment of the method according to the present invention, a parallel hybridization-based real-time quantification of PCR targets can be achieved. In this case, an asymmetrical PCR is performed according to one of the embodiments mentioned above. Contrarily to the embodiments already described, the continuous amplification and hybridization process is performed cyclically, however. I.e. a hybridization is performed subsequently to every PCR cycle or respectively to a certain number of PCR cycles. The hybridization signals measured at the individual probes are quantified after each hybridization step. These signals are a measure for the concentration of the target in the reaction solution at the moment of the hybridization reaction.

By means of the multiplicity of probes on the probe array, a multiplicity of targets can be quantified simultaneously. Amplification kinetics of the individual targets can be traced by tracing the increase of the hybridization signals measured at each spot as the number of cycles increases.

Naturally, the cyclical continuous PCR and hybridization reaction can be performed by means of using structure breaker molecules according to one of the preceding examples or without their addition.

Summoned up, these methods present the possibility of a faster performance of microarray-based analyses. Analyses are reduced to a few steps, which reduces the susceptibility for mistakes. Analyses comprising an amplification and hybridization of targets can now be performed entirely in closed systems like for example the assay processor by Clondiag, which is described, inter alia, in the International Patent Application WO 01/02094. Thereby, a substantial precondition is provided for performing array-based analyses of nucleic acids by means of small, handy, optionally mobile systems, so that a basis for chip-based ‘point of care’-diagnostics is created for the first time. Furthermore, working in closed systems reduces the danger of contamination with possibly infectious sample material and the transfer of amplification products to other reactions.

In the following, the invention is explained by way of concrete examples. These are not to be interpreted in a restrictive manner.

### EXAMPLES

### Example 1

Comparative Hybridization of PCR Products Produced by Means of Symmetrical PCR and Asymmetrical PCR According to the Present Invention with Graded Competitor Proportions Against Immobilized Probes.

Amplification Kinetics of the Asymmetrical PCR:

In an initial experiment it was first examined, to which extent the product formation in the PCR depends on the competitor proportion. To this end, PCR reactions with identical primer concentrations but having different competitor proportions were performed first.

The competitor was a DNA oligonucleotide having the same sequence as the reverse primer of the PCR but being modified at the 3′-OH group at its 3′-end with an NH₂-group. The amino modification was integrated during the chemical synthesis of the oligonucleotide into the molecule (3′-amino-modifier C7, Glen Research Corp., Sterling, Va., USA).

PCR Reactions:

The forward primer 16sfD1Cy3 had the following sequence and was labeled with Cy3 at its 5′-end.

5′-AGAGTTTGATCGTGGCTCAG-3′

The reverse primer had the following sequence:

5′-TACCGTCACCATAAGGCTTCGTCGCTA-3′

PCR setups having different competitor proportions were prepared. Herein, each PCR setup had the following composition and final concentrations.

| | |
|---|---|
| 1 x | PCR reaction buffer (Eppendorf Hamburg, Germany) |
| 200 nM | forward primer 16sfD1Cy3, labeled with the fluorescent |
| | colorant Cy3 (Amersham-Pharmacia, Freiburg, Germany) at |
| | its 5′-end |
| 200 μM | dNTPs |
| 0.05 U/μl | Taq polymerase (Eppendorf Hamburg, Germany) |
| 2 ng/μl | chromosomal DNA Corynebacterium glutamicum |

additional contents were:

| | | |
|---|---|---|
| Reaction 1: | 200 nM | reverse primer 16s Ra (corresponding to 0% |
| | | competitor, symmetrical PCR) |
| Reaction 2: | 0 nM | reverse primer 16s Ra |
| | 200 nM | competitor 16s Ra 3′ NH2 (corresponding to |
| | | 100% competitor, asymmetrical PCR) |
| Reaction 3: | 10 nM | reverse primer 16s Ra |
| | 190 nM | competitor 16s Ra 3′ NH2 (corresponding to 95% |
| | | competitor, asymmetrical PCR) |
| Reaction 4: | 20 nM | reverse primer 16s Ra |
| | 180 nM | competitor 16s Ra 3′ NH2 (corresponding to 90% |
| | | competitor, asymmetrical PCR) |
| Reaction 5: | 40 nM | reverse primer 16s Ra |
| | 160 nM | competitor 16s Ra 3′ NH2 (corresponding to 80% |
| | | competitor, asymmetrical PCR) |
| Reaction 6: | 60 nM | reverse primer 16s Ra |
| | 140 nM | competitor 16s Ra 3′ NH2 (corresponding to 70% |
| | | competitor, asymmetrical PCR) |
| Reaction 7: | 100 nM | reverse primer 16s Ra |
| | 100 nM | competitor 16s Ra 3′ NH2 (corresponding to 50% |
| | | competitor, asymmetrical PCR) |
| Reaction 8: | 140 nM | reverse primer 16s Ra |
| | 60 nM | competitor 16s Ra 3′ NH2 (corresponding to 30% |
| | | acompetitor, symmetrical PCR) |

The total volume of each PCR setup was 25 μl. The reactions were performed according to the following temperature regime:

| | | |
|---|---|---|
| Initial denaturation: | 2 min | 95° C. |
| 25 cycles: | 30 sec | 95° C. |
| | 30 sec | 60° C. |
| | 30 sec | 72° C. |
| Terminal elongation: | 7 min | 72° C. |

The reaction setups were filled into LightCycler cuvettes (Roche Diagnostics, Mannheim, Germany) and the PCR reaction was performed in the LightCycler (Roche Diagnostics, Mannheim, Germany). Product formation kinetics were recorded according to the manufacturer's instructions.

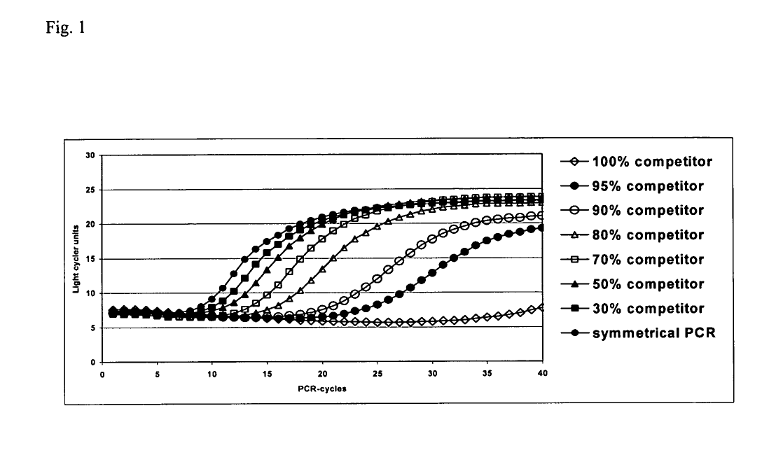

The results are depicted in FIG. 1 (FIG. 1). Herein, the amount of PCR product formed is given as LightCycler units depending on the number of cycles.

It can distinctly be seen that the efficiency of the PCR and correspondingly the product yield decreases as the competitor proportion increases.

Comparison of the Hybridization Signals Depending on the Competitor Proportion:

In the following, product formation and hybridization signal were compared in the cases of symmetrical and asymmetrical PCR amplification with different competitor proportions at a certain point in time (completion of the reaction after 25 cycles).

The same primers and competitors, respectively, as specified above were used.

PCR Reactions:

PCR setups with different competitor proportions were prepared. Herein, each PCR setup had the following composition and final concentrations.

| | |
|---|---|
| 1 x | PCR reaction buffer (Eppendorf Hamburg, Germany) |
| 200 nM | forward primer 16sfD1Cy3, labeled with the fluorescent |
| | colorant Cy3 at its 5′-end (Amersham-Pharmacia, Freiburg, |
| | Germany) |
| 200 μM | dNTPs |
| 0.05 U/μl | Taq polymerase (Eppendorf Hamburg, Germany) |
| 2 ng/μl | chromosomal DNA Corynebacterium glutamicum |

additional contents were:

| | | |
|---|---|---|
| Reaction 1: | 200 nM | reverse primer 16s Ra (corresponding to 0% |
| | | competitor, symmetrical PCR) |
| Reaction 2: | 80 nM | reverse primer 16s Ra |
| | 120 nM | competitor 16s Ra 3′ NH2 (corresponding to |
| | | 60% competitor, asymmetrical PCR) |
| Reaction 3: | 40 nM | reverse primer 16s Ra |
| | 160 nM | competitor 16s Ra 3′ NH2 (corresponding to |
| | | 80% competitor, asymmetrical PCR) |
| Reaction 4: | 20 nM | reverse primer 16s Ra |
| | 180 nM | competitor 16s Ra 3′ NH2 (corresponding to |
| | | 90% competitor, asymmetrical PCR) |

The reactions were performed according to the same temperature protocol as above:

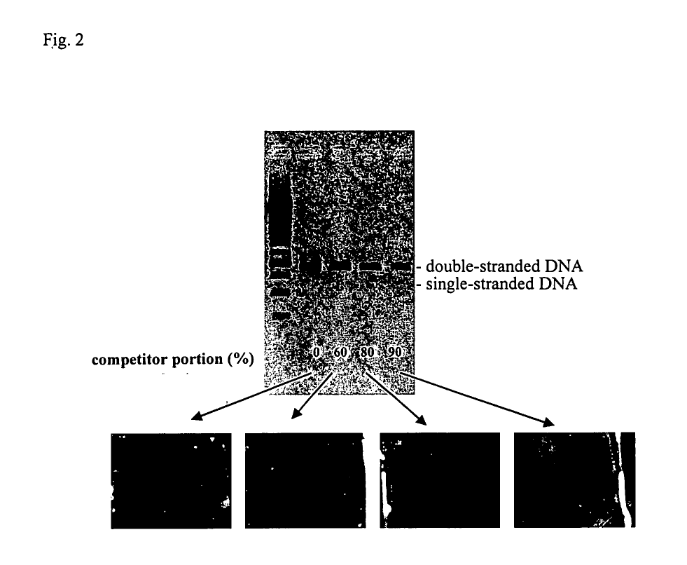

The total volume of each PCR was 25 μl. 5 μl of each of the PCR setups were analyzed on a 2% agarose gel (FIG. 2). It can be seen that the amount of double-stranded DNA decreases and the amount of single-stranded DNA increases as the competitor proportion increases.

Subsequently, the remaining 20 μl of the PCR reactions were purified by means of the Quiaquick PCR purification kit (Qiagen, Hilden, Germany) according to the manufacturer's instructions. The elution of the PCR fragments was performed in 50 μl water in each case. The eluate was constricted to 10 μl in a vacuum.

Immobilization of the Hybridization Probes:

On glass surfaces of the size of 3×3 mm (chip), which were coated with an epoxide, an amino-modified oligonucleotide with a length of 18 nucleotides of the sequence 5′-NH₂-GTTTCCCAGGCTTATCCC-3′) was covalently immobilized at two defined places (spot).

For this purpose 0.1 μl of a 5 μM solution of the oligonucleotide in 0.5 M phosphate buffer were applied on the glass surface and eventually dried at 37° C. The covalent linking of the applied oligonucleotides with the epoxide groups on the glass surface was performed by 30 min baking of the chips at 60° C. Subsequently, the chips were vigorously rinsed with distilled water and then washed for 30 min in 100 mM KCl. After further short rinsing in 100 mM KCl and subsequently in distilled water, the chips were dried for 10 min at 37° C.

Hybridization:

2 μl of the purified PCR aliquots were taken up in 50 μl 6×SSPE, 0.1% SDS (Sambrook et al., vide supra). A chip was added to each hybridization solution. The reaction was denatured for 5 min at 95° C. and subsequently incubated for 1 h at 50° C. Subsequently, 3 consecutive 5-min washing steps at 30° C. in 2×SSC, 0.1% SDS, at 30° C. in 2×SSC and at 20° C. in 0.2×SSC were performed (Sambrook et al. vide supra). The chips were subsequently dried in a vacuum.

Detection of the Hybridization Signals:

The detection of the hybridization signals was performed under a Zeiss fluorescence microscope (Zeiss, Jena, Germany). The stimulation was performed in incident light with a white light source and with a set of filters suitable for Cyanine 3. The signals were recorded with a CCD camera (PCO-Sensicam, Kehlheim, Germany). Exposure time was 2000 ms. (FIG. 2)

Results:

It showed that the PCR products produced according to the method of the present invention exhibited significantly increased hybridization signals despite a significantly decreased amount of product.

As the competitor concentration increases within a range of 0 to 90% competitor proportion, an increase of the hybridization signal can be observed although the total amount of product decreases. Surprisingly, the best signals were achieved with the highest competitor proportion (90%) and therefore with the smallest amount of single-stranded DNA.

### Example 2

A quantitative analysis of the hybridization signal depending on the competitor proportion was performed. The same forward and reverse primers and the same competitor as in Example 1 were used for the PCR.

PCR setups having the following composition and final concentrations were prepared:

all reactions contained:

| | |
|---|---|
| 1 x | PCR reaction buffer (Eppendorf Hamburg, Deutschland) |
| 200 nM | forward primer 16sfD1Cy3, labeled with the fluorescent |
| | colorant Cy3 (Amersham-Pharmacia, Freiburg, Germany) at |
| | its 5′-end |
| 200 μM | dNTPs |
| 0.05 U/μl | Taq polymerase (Eppendorf Hamburg, Germany) |
| 2 ng/μl | chromosomal DNA Corynebacterium glutamicum |

additional contents were:

| | | |
|---|---|---|
| Reaction 1: | 266 nM | reverse primer 16s Ra, no competitor |
| | | (symmetrical PCR) |
| Reaction 2: | 133 nM | reverse primer 16s Ra |
| | 133 nM | competitor 16s Ra 3′ NH2 (corresponding to 50% |
| | | competitor, asymmetrical PCR) |
| Reaction 3: | 40 nM | reverse primer 16s Ra |
| | 160 nM | competitor 16s Ra 3′ NH2 (corresponding to 75% |
| | | competitor, asymmetrical PCR) |
| Reaction 4: | 33 nM | reverse primer 16s Ra |
| | 233 nM | competitor 16s Ra 3′ NH2 (corresponding to |
| | | 87.5% competitor, asymmetrical PCR) |

The reactions were divided into aliquots of 25 μl each. The temperature protocol was identical to Example 1. One aliquot of each of the reactions 1-4 was withdrawn after 15, 20, 25, 30, and 35 PCR cycles, respectively.

5 μl of each aliquot were analyzed on a 2% agarose gel in each case. Gel analysis showed that significantly more product was produced with a small number of cycles in a symmetrical PCR than in the asymmetrical PCR reaction.

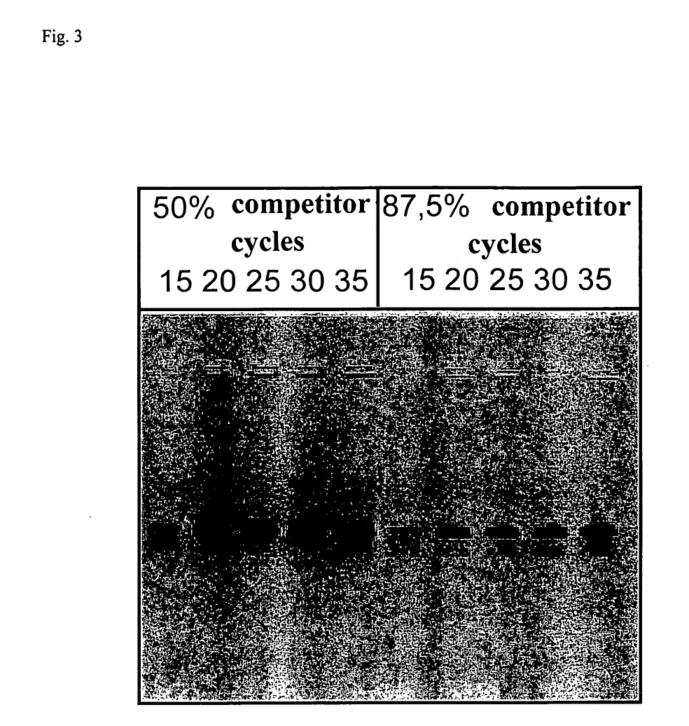

FIG. 3 (FIG. 3) shows an agarose gel, on which 5 μl-samples of reactions having a competitor proportion of 50% and 87.5%, respectively, were analyzed parallel. The reactions had each been stopped after the given number of cycles. It can be seen that less product is produced with 87.5% competitor proportion.

Immobilization of the Hybridization Probes:

On glass surfaces of the size of 3×3 mm (chip), which were coated with an epoxide, an amino-modified oligonucleotide with a length of 18 nucleotides of the sequence 5′-NH₂-GTTTCCCAGGCTTATCCC-3′) was covalently immobilized at two defined places (spot).

In addition thereto, 0.1 μl of a 5 μM solution of the oligonucleotide in 0.5 M phosphate buffer were applied on the glass surface and eventually dried at 37° C. The covalent link of the applied oligonucleotides with the epoxide groups on the glass surface was performed by 30 min baking of the chips at 60° C. Subsequently, the chips were vigorously rinsed with distilled water and then washed for 30 min in 100 mM KCl. After further short rinsing in 100 mM KCl and subsequently in distilled water, the chips were dried for 10 min at 37° C.

Hybridization:

Further 5 μl of the reaction were mixed with 50 μl 6×SSPE, 0.1% SDS and employed in hybridization experiments (Sambrook et al., vide supra). A chip was added to each hybridization solution. The reaction was denatured for 5 min at 95° C. and subsequently incubated for 1 h at 50° C. Subsequently, 3 consecutive 5-min washing steps at 30° C. in 2×SSC, 0.1% SDS, at 30° C. in 2×SSC and at 20° C. in 0.2×SSC were performed. The chips were subsequently dried in a vacuum.

Detection of the Hybridization Signals:

The detection of the hybridization signals was performed under a Zeiss fluorescence microscope (Zeiss, Jena, Germany). The stimulation was performed in incident light with a white light source and with a set of filters suitable for Cyanine 3. The signals were recorded with a CCD camera (PCO-Sensicam, Kehlheim, Germany). Exposure time was 2000 ms.

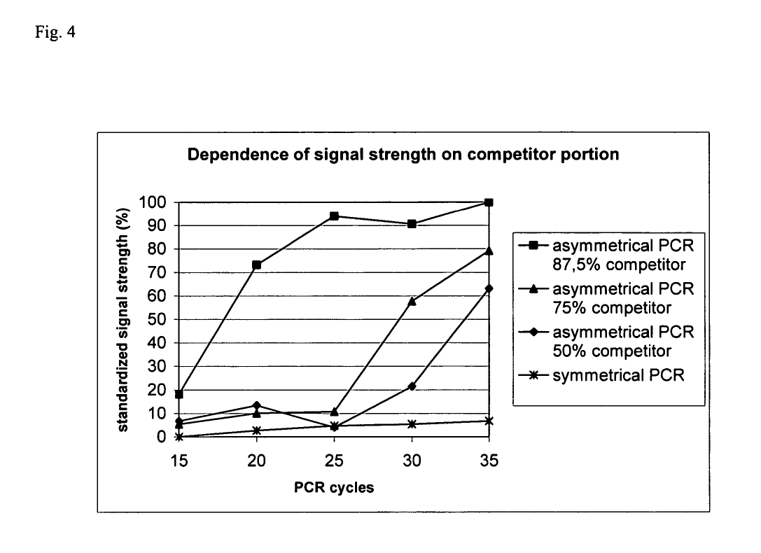

The intensity of the hybridization signals was measured by averaging the signal strength (measured shade of gray) over the entire region of the spots. The shade of gray value averaged over the spot-free region (background) was subtracted from this value. The signal intensities calculated this way were standardized (highest value=100%) and plotted against the number of cycles (FIG. 4).

Results:

FIG. 4 shows that a stronger hybridization signal is achieved by means of PCR products from asymmetrical PCR reactions over the entire examined range of 15 to 35 cycles, although the gel analysis (FIG. 3) for these reactions showed a smaller amount of product, in particular after 15 and 20 cycles.

The strongest signal was surprisingly achieved with the highest competitor proportion, although herein the smallest amount of single-stranded target molecules was present. The hybridization of PCR products produced by means of asymmetrical PCR with varying competitor proportion therefore showed a hybridization signal up to 20 times higher over the entire measurable range of the amplification.

### Example 3

### Detection of the Formation of a Single Strand Surplus in Asymmetrical PCR

Two PCR setups (150 μl each) having the following composition and final concentrations were prepared.

The forward and the reverse primer as well as the competitor had the same sequences as in Example 1. However, the forward primer was labeled with the fluorescent colorant IRD 800 at its 5′-end.

Both reactions contained:

| | |
|---|---|
| 1 x | PCR reaction buffer (Eppendorf Hamburg, Germany) |
| 200 nM | forward primer 16sfD1IRD, labeled with the fluorescent |
| | colorant IRD 800 (MWG-Biotech, Ebersberg, Germany) at |
| | its 5′-end |
| 160 μM | dNTPs |
| 0.1 U/μl | Taq polymerase (Eppendorf Hamburg, Germany) |
| 2 ng/μl | chromosomal DNA Corynebacterium glutamicum ATCC 13032 |

additional contents were:

| | | |
|---|---|---|
| Reaction 1: | 200 nM | reverse primer 16s Ra, no competitor |
| | | (symmetrical PCR) |
| Reavtion 2: | 20 nM | reverse primer 16s Ra |
| | 180 nM | competitor 16s Ra 3′ NH2 (corresponding to 90% |
| | | competitor, asymmetrical PCR) |

The reactions were divided into 6 aliquots of 25 μl each (1/a -1/f and 2/a -2/f respectively) and incubated according to the following temperature protocol:

| | | |
|---|---|---|
| Initial denaturation | 2 min | 95° C. |
| 35 cycles | 30 sec | 95° C. |
| | 30 sec | 50-70° C. |
| | | correspondingly for: |
| | | Reaction a: 50° C. |
| | | Reaction b: 54° C. |
| | | Reaction c: 60° C. |
| | | Reaction d: 63° C. |
| | | Reaction e: 67° C. |
| | | Reaction f: 70° C. |
| | 30 sec | 72° C. |
| Terminal extension | 7 min | 72° C. |

Herein, the annealing temperatures for the reactions e and f were selected in such a way that an annealing of the competitor and the reverse primer, respectively, was not to take place, as the temperatures were higher than the melting temperatures of these oligonucleotides.

2 μl of a 1:10 dilution of each aliquot were analyzed on a 3.5% native polyacrylamide gel in a LICOR sequencer. The gel had a thickness of 1 mm. The electrophoresis was performed with a limitation of voltage to 200 V and a limitation of power to 5 W. The gel was neither actively heated nor actively cooled (The control variable for temperature was set at 15° C.).

Data recording was performed with a depth of 16 bit, scan speed 1 and signal filter 3.

During the PCR amplification, only one of the two strands is labeled with the IRD colorant. Only this strand is visible in the gel analysis in the LICOR sequencer. As a native gel was used, the PCR products are separated not only according to length, but also according to structural properties.

Results:

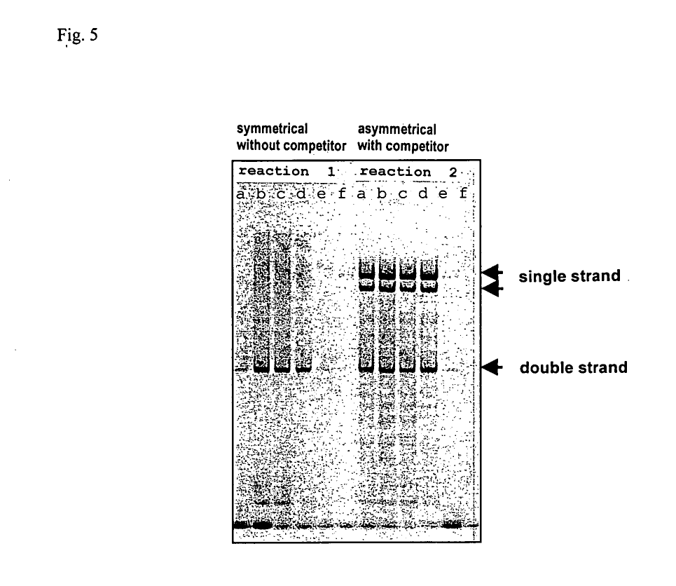

The results are shown in FIG. 5. As expected, only one dominating product is detected in the gel analysis of the symmetrical reactions for all reactions, wherein an amplification took place. This is the double-stranded PCR product. Contrarily to this, 3 dominating products are detected in all detectable asymmetrical reactions. Two further bands corresponding to different structures of the labeled single strand are obtained beside the double-stranded template. Therefore, the gel analysis shows that the asymmetrical amplification leads to a single strand surplus.

### Example 4

Increase of the Hybridization Signal by Addition of Secondary Structure Breakers

An asymmetrical multiplex PCR reaction was performed, which had the following composition and final concentrations. Genomic DNA, which contained the human cyp2D6 gene and whose genotype was known, was used as template:

1 μl cyp2D6 fusion primer mixture consisting of:

0.5 μM uniA_cyp2D6_1/2f
(5′GGAGCACGCTATCCCGTTAGACCAGAGGAGCCCATTTGGTAGTGAGG
CAGGT3′)
1 μM uniA_cyp2D6_1/2f_F2
(5′GGAGCACGCTATCCCGTTAGACTGGACGCCGGTGGTGGTGCTCAA
3′)
1.5 μM uniA_cyp2D6_3/4f
(5′GGAGGACGGTATCCCGTTAGACCACGCGCACGTGCCGGTCCCA3′)
1 μM uniA_cyp2D6_5/6f_kF
(5′GGAGCACGCTATCCCGTTAGACCGCTGGCTGGCAAGGTCCTACG
C3′)
0.8 μM uniA_cyp2D6_8/9f_kF
(5′GGAGCACGCTATCCCGTTAGACGCCCCGGCCCAGCCACCAT3′)
1 μM uniB_cyp2D6_1/2r
(5′CGCTGCGAACTACCGCACATGGGTCCCACGGAAATCTGTCTCTGT
3′)
0.5 μM uniB_cyp2D6_1/2r_F1
(5′CGCTGCCAACTACGGCACATGCCTCTGGCGCCCTGCAGGACCTC3′)
1.5 μM uniB_cyp2D6_3/4r
(5′CGCTGCCAACTACCGCACATGCTCTCGCTCCGCACCTCGCGCAGA
3′)
1 μM uniB_cyp2D6_5/6r
(5′CGCTGCCAACTACCGCACATGCCCTCGGCCCCTGCACTGTTTCCC
AGA3′)
0.8 μM uniB_cyp2D6_8/9r_kF
(5′CGCTGCCAACTAGCGCACATGTGGCTAGGGAGCAGGCTGGGGAC
T3′)

| | |
|---|---|
| 200 μM | dNTPs |
| 1× | Advantage PCR reaction buffer (Clontech, Palo Alto, USA) |
| 0.1 U/μl | Advantage DNA polymerase (Clontech, Palo Alto, USA) |
| 1 μl | human DNA sample KDL 36 (0.71 μg/μl) (Urs Meyer, |
| | Biozentrum Basel, Switzerland) genotype: homozygous cyp2D6 |
| | C2938T |

The total volume of the PCR was 25 μl. In the now following first phase of the two-phase amplification reaction, the incubation was performed under the following conditions:

| | | |
|---|---|---|
| Initial denaturation: | 95° C. | 10 min |
| 25 cycles: | 95° C. | 35 sec |
| | 65° C. | 50 sec |
| | 72° C. | 70 sec |

Cooling down to 4° C.

During the course of the reaction, the primers were used up and all fragments of the multiplex PCR were amplified to a uniform molar level. In this first phase, the amplification was performed with primers, whose 3′-end had specificity for the target fragments, whose 5′-ends, however, had a uniform sequence for all forward primers and for all reverse primers.

In the now following second phase of the reaction, primers being complementary to the uniform sequences were added. Simultaneously, a competitor having the same sequence as the now employed reverse primer but having a NH₂ modification at its 3′-OH group at its 3′-end was added. Thereby, the reaction in the second phase was performed asymmetrically.

The universal forward primer uniB had the following sequence and was labeled with a Cy3 group at its 5′-end:

5′-GGAGCACGCTATCCCGTTAGAC-3′

The universal reverse primer uniA had the following sequence:

The total volume of the PCR reaction was 25 μl. In the second phase, the reaction was incubated as follows:

| | | |
|---|---|---|
| Initial denaturation: | 95° C. | 30 sec |
| 20 cycles: | 95° C. | 35 sec |
| | 65° C. | 50 sec |
| | 72° C. | 90 sec |
| Terminal elongation: | 72° C. | 7 min |

### Array Layout

A probe array was produced by means of in situ synthesis. The array consisted of 1,024 probe elements of 64×64 μm in size. These spots represented two different oligonucleotides:

| | |
|---|---|
| C2938T | 5′AATGATGAGAACCTGTGCATAGTGGTG 3′ |
| C2938 WT | 5′AATGATGAGAACCTGCGCATAGTGGTG 3′ |

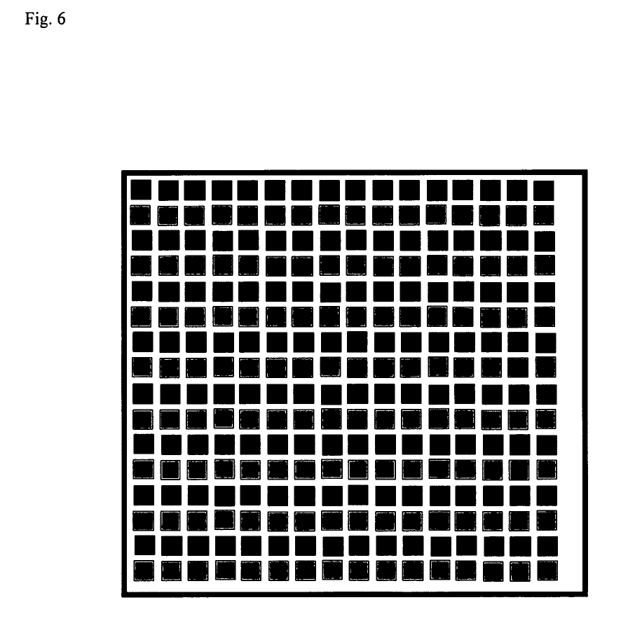

The probes were arranged according to the pattern depicted in FIG. 6. Black fields represent the probe C2938T, gray fields represent the probe C2938 WT.

Hybridization:

5 hybridization setups were prepared by taking up 5 μl of the multiplex PCR reactions in 60 μl SSPE, 0.1% SDS in each case. The structure breakers

| | |
|---|---|
| C2938TBL3 | (5′-GGCTGACCTGTTCTCTGCCG-3′) |
| and | |
| C2938TBL5 | (5′-GGAACCCTGAGAGCAGCTTC-3′) |

were added in different molar concentrations:

| | |
|---|---|
| Reaction 1: | no structure breakers |
| Reaction 2: | 1.5 nM structure breaker each |
| Reaction 3: | 15 nM structure breaker each |
| Reaction 4: | 150 nM structure breaker each |
| Reaction 5: | 1.5 μM structure breaker each |

An array having the above-described layout was added to each reaction. The hybridization reactions were denatured for 5 min at 95° C. and subsequently incubated for 1 h at 50° C. Subsequently, 3 consecutive 5-min washing steps at 30° C. in 2×SSC, 0.1% SDS, at 30° C. in 2×SSC and at 20° C. in 0.2×SSC were performed. The chips were withdrawn and subsequently dried in a vacuum.

Detection of the Hybridization Signals:

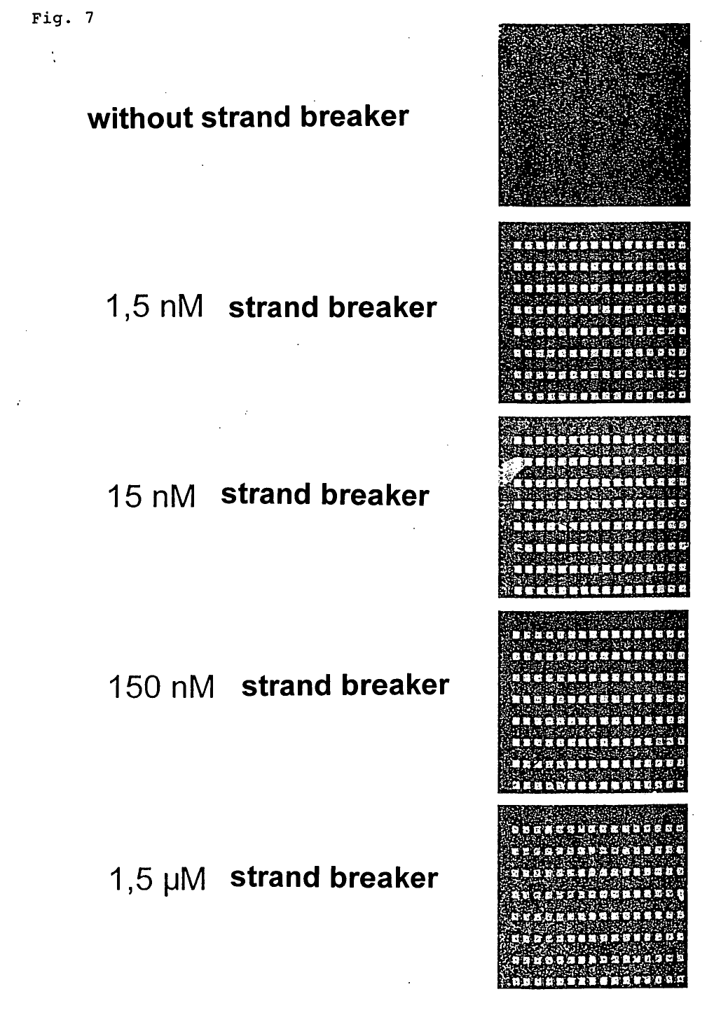

The detection of the hybridization signals was performed under a Zeiss fluorescence microscope (Zeiss, Jena, Germany). The stimulation was performed in incident light with a white light source and with a set of filters suitable for Cyanine 3. The signals were recorded with a CCD camera (PCO-Sensicam, Kehlheim, Germany). Exposure time was 5000 ms. (FIG. 7)

Results:

The hybridization results exhibited the following pattern: The probes for the mutation C2938T exhibited strong signals, while the wild type probes and the detection variants exhibited significantly lower signals. This is corresponding to the expectations. It is ostentatious that the signals are further enhanced by addition of structure breakers. Therefore, a combination of competitor and secondary structure breaker is particularly useful for achieving good signal strengths.

### Example 5

Addition of a Secondary Structure Breaker Hybridizing in the Proximity of Sequence Portions of the Target, which are Complementary to the Probes.

An asymmetrical multiplex PCR reaction identical to the one in Example 4 was performed. Arrays having the same layout as in the preceding Example 4 were used.

Hybridization:

4 hybridization setups were prepared by means of taking up 5 μl of the multiplex PCR reactions in 60 μl SSPE, 0.1% SDS in each case. The hybridization setups differed concerning the addition of the structure-breakers

| | |
|---|---|
| C2938TBL3 | (5′-GGCTGACCTGTTCTCTGCCG-3′) bzw. |
| C2938TBL5 | (5′-GGAACCCTGAGAGCAGCTTC-3′). |

| | |
|---|---|
| Reaction 1: | no structure breakers |
| Reaction 2: | 1.5 μM structure breaker C2938TBL3 |
| Reaction 3: | 1.5 μM structure breaker C2938BL5 |
| Reaction 4: | 1.5 μM structure breaker C2938TBL3 and C2938TBL5 |
| | each |

An array having the above-described layout was added to each reaction. The hybridization reactions were denatured for 5 min at 95° C. and subsequently incubated for 1 h at 50° C. Subsequently, 3 consecutive 5-min washing steps at 30° C. in 2×SSC, 0.1% SDS, at 30° C. in 2×SSC and at 20° C. in 0.2×SSC were performed. The chips were withdrawn and subsequently dried in a vacuum.

Detection of the Hybridization Signals:

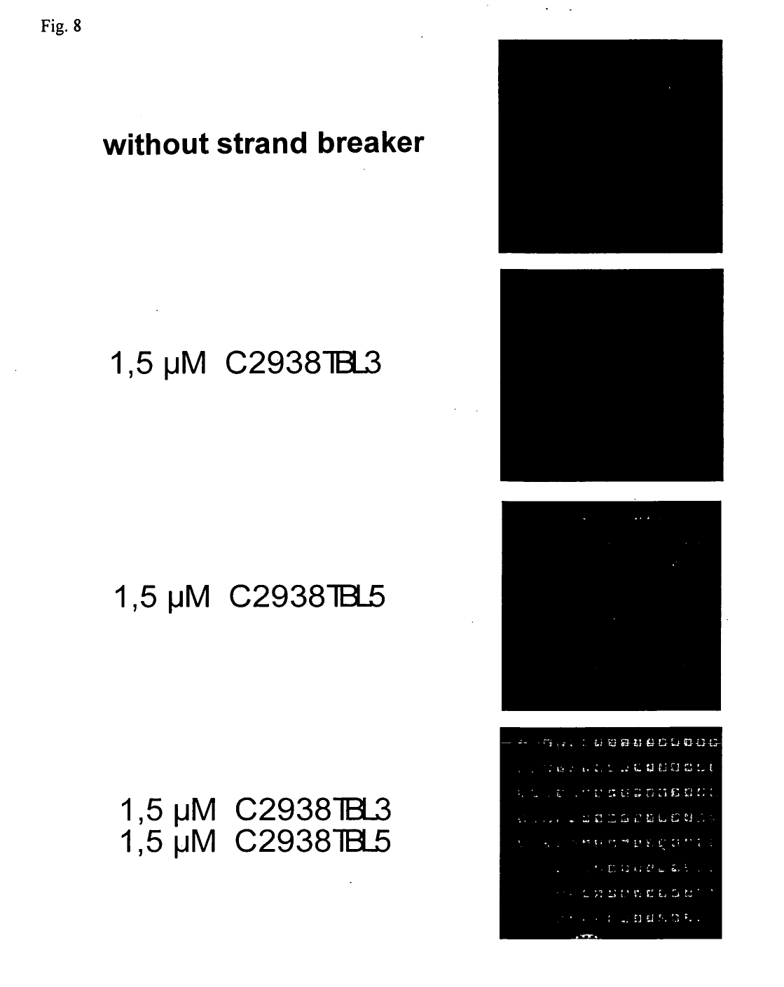

The detection of the hybridization signals was performed under a Zeiss fluorescence microscope (Zeiss, Jena, Germany). The stimulation was performed in incident light with a white light source and with a set of filters suitable for Cyanine 3. The signals were recorded with a CCD camera (PCO-Sensicam, Kehlheim, Germany). Exposure time was 10000 ms (FIG. 8).

Results:

The hybridization results exhibited the following pattern: The probes for the mutation C2938T exhibited strong signals, while the wild type probes and the detection variants exhibited significantly lower signals. This is corresponding to the expectations. It is ostentatious that significantly higher signals are already achieved when adding one of the two structure breakers.

### Example 6

Influence of Structure Breakers on the Match-Mismatch Discrimination

In the present example, secondary structure breakers are oligonucleotides binding near the actual hybridization probe in the hybridization of a double-stranded nucleic acid. Presumably, the double strand of the nucleic acid is disintegrated at the respective site and the binding capability of the hybridization probe is improved. As shown in the following example, an enhancement of the hybridization signal by the factor 3 to 5 thereby takes place without influencing the specificity of the reaction.

Preparation of the Array:

397 DNA probe arrays, each having 15 oligonucleotide probes of different sequence and length, were synthetically produced on an epoxidized glass wafer by Schott. All probes were complementary to a partial sequence of the exon 5/6 of the human cyp2D6 gene and differed only in the mid-region of the probe due to addition of several different mutations.

Subsequently to the synthesis, the wafer was sawed up into chips the size of 3.4×3.4 mm, wherein each of these chips contained a probe array with all 15 oligonucleotides in different redundancies. Altogether, each array consisted of 256 spots. These were arranged in 16 identical fields of 16 spots. The following sequences were set up in the individual spots.

| | |
|---|---|
| C2938T | AATGATGAGAACGTGTGCATAGTGGTG |
| C2938TC | AATGATGAGAACCTGTCGCATAGTGGTG |
| C2938GT | AATGATGAGAAGCTGGTGCATAGTGGTG |
| C2938GTC | AATGATGAGAACCTGGTCGCATAGTGGTG |
| C2938G | AATGATGAGAACCTGGGCATAGTGGTG |
| G2938GC | AATGATGAGAACCTGGCGCATAGTGGTG |
| C2938d | AATGATGAGAACCTGGCATAGTGGTG |
| C2938CT | AATGATGAGAACCTGCTGCATAGTGGTG |
| C2938CTC | AATGATGAGAACCTGCTCGCATAGTGGTG |
| C2938 | AATGATGAGAACCTGCGCATAGTGGTG |
| C2938CC | AATGATGAGAACCTGCCGCATAGTGGTG |
| C2938AT | AATGATGAGAACGTGATGCATAGTGGTG |
| C2938ATC | AATGATGAGAACCTGATCGCATAGTGGTG |
| C2938A | AATGATGAGAACCTGAGCATAGTGGTG |
| C2938AC | AATGATGAGAACCTGACGCATAGTGGTG |

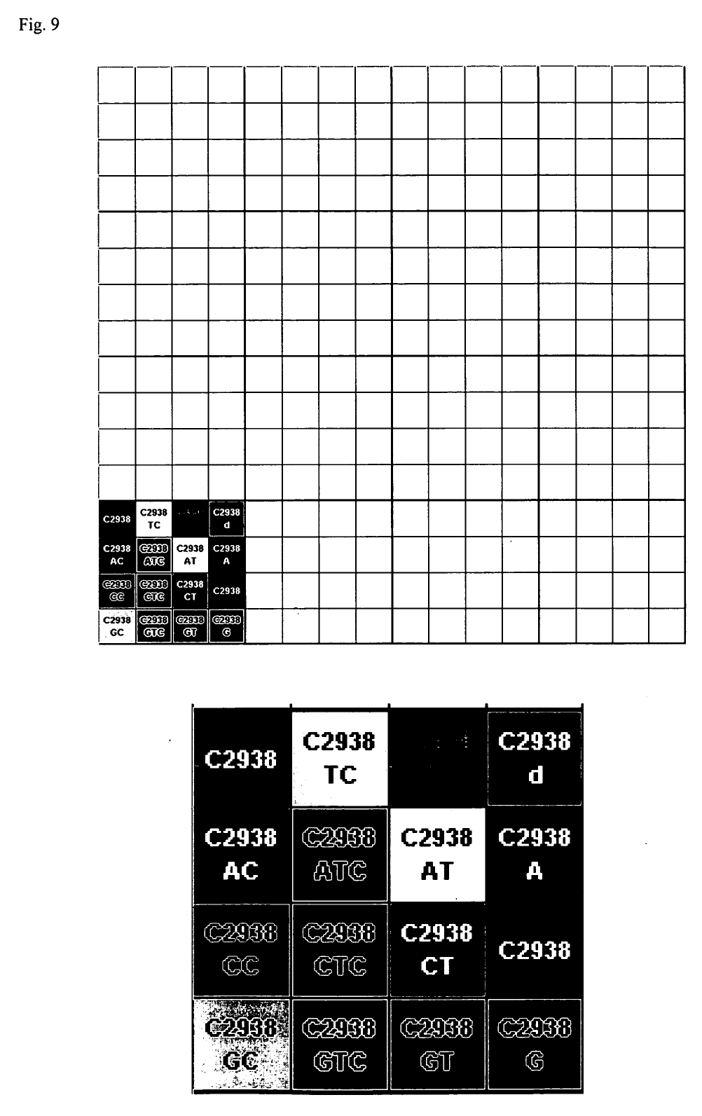

The arrangement of the probes on the array was performed as shown in FIG. 9. Each of the 16 thick-lined squares contained 16 array elements equipped with the probes shown for the square in the lower left portion of the illustration.

Preparation of the DNA Sample to be Hybridized:

For the amplification of the target for the hybridization, an asymmetrical PCR with a clinical DNA sample as template (KDL 31, provided by Prof. U. Meyer, Biozentrum Basel, Switzerland) was performed. By means of the PCR, a partial sequence of the exon 5/6 of the human cyp2D6 gene was amplified. In the PCR, a competitor, whose sequence was identical to the forward primer but was modified with an NH₂ group at the 3′-OH group at its 3′-end, was employed. The reverse primer was labeled with Cy3 at its 5′-end.

To this end, a PCR setup with the following final concentrations was prepared according to the following scheme:

| | |
|---|---|
| 20 nM | forward primer: cyp2D6_5/6f |
| | (5′GGACTCTGTACCTCCTATCCACGTCA3′) |
| 180 nM | competitor: cyp2D6_5/6f_3NH2 |
| | (5′GGACTCTGTACCTCCTATCCACGTCA-NH2-3′) |
| 200 nM | reverse primer: cyp2D6_5/6r_5Cy3 |
| | (5′-Cy3-CCCTCGGCCCCTGCACTGTTTCCCAGA3′) |
| 200 μM | dNTPs |
| 1 x | 10 x Taq reaction buffer (Eppendorf AG, |
| | Hamburg, Germany) |
| 5 U | MasterTaq DNA polymerase (Eppendorf AG, |
| | Hamburg, Germany), 1 ng/μl template DNA |
| | (KDL 31), Concentration 50 ng/μl) |

The total volume of the PCR was 50 μl.

This setup was incubated according to the following temperature protocol:

| | | |
|---|---|---|
| Initial denaturation: | 10 min | 95° C. |
| 30 cycles: | 30 sec | 95° C. |
| | 50 sec | 62° C. |
| | 90 sec | 72° C. |
| Terminal elongation | 7 min | 72° C. |

The reaction products were purified via columns (PCR Purification Kit, Qiagen, Hilden, Germany) and subsequently quantified according to the manufacturer's protocol.

Hybridization:

In order to examine the influence of the secondary structure breaker on the hybridization, two different hybridization setups were performed: The corresponding secondary structure breakers were added to the first setup, the second setup served as control having no secondary structure breaker.

To this end, the Cy3-labeled asymmetrical PCR was taken up in a final concentration of 25 nM in 50 μl 6×SSPE, 0.1% SDS. In addition, the structure breakers

2938BL5 (5′-GGAACCCTGAGAGCAGCTTC-3′)
and
2938BL3 (5′-GGCTGACCTGTTGTCTGCCG-3′)

were added to setup 1 in a final concentration of 1 μM each.

After addition of a chip to each hybridization setup, they were denatured for 5 min at 95° C. and then incubated for 1 h at 45° C. Subsequently, while being shaken, the chips were washed for 10 min at 30° C. in 2×SSC, 0.2% SDS, at 30° C. in 2×SSC and at 20° C. in 0.2×SSC (Sambrook et al., vide supra) and blow-dried by means of compressed air.

Detection of the Hybridization:

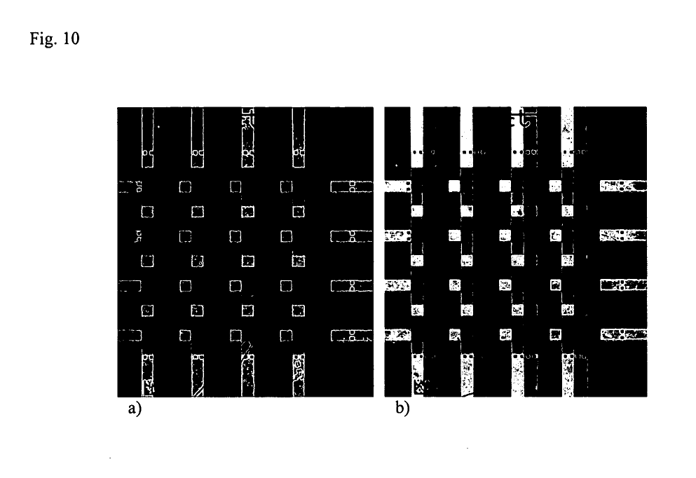

The hybridized and washed chips were read out in a slide scanner (Scanarray4000, GSI Lumonics). Herein, the chips were recorded with a scanner setting offering a region as dynamic as possible with the prevailing signal intensity. In addition, a fluorescence standard (Fluoris®I, Clondiag Chip Technologies) was recorded with the selected measurement settings for later standardization of the data (FIG. 10).

Results:

The results of the hybridization are depicted in FIG. 10. FIG. 10a shows the fluorescence signals after hybridization of a PCR with the addition of structure breakers. The recording was made in the slide scanner with Laser-Power 70 and Photomultiplier 80. FIG. 10b shows the negative control, however, without addition of the corresponding structure breakers. The scanner settings were Laser-Power 100 and Photomultiplier 75. It is to be noted that FIG. 10b was thus recorded with a significantly higher laser and photomultiplier performance of the scanner. Therefore, the signals on this chip only appear to be stronger.

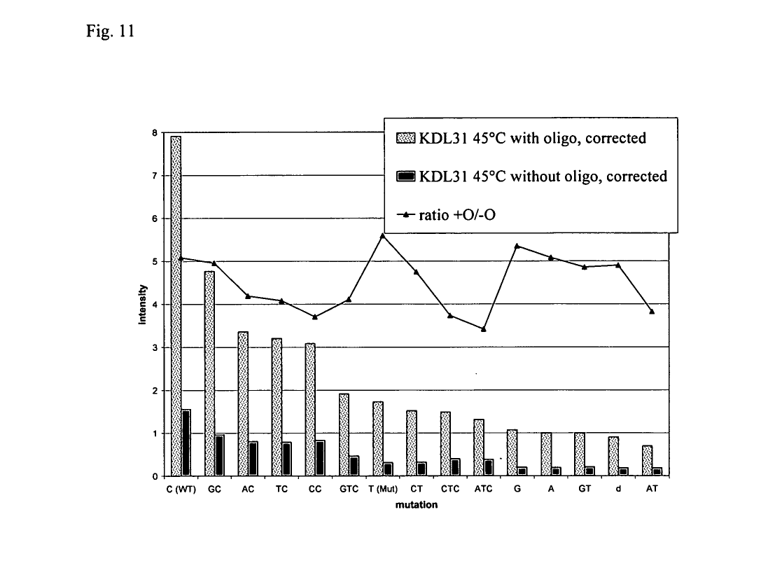

The recorded pictures were evaluated by means of the picture evaluation software Iconoclust® (Clondiag Chip Technologies) and the obtained data were standardized by means of the data from the fluorescence standard. The measuring values thus obtained are plotted in the diagram in FIG. 11.

In FIG. 11, it can be seen that the addition of structure breakers causes an increase of the fluorescence signal by the factor 3.5 to 5.5. As can be seen from the ratio of the hybridization signals with and without secondary structure breaker oligonucleotide, the specificity of the hybridization independent of the examined mutation is not influenced within the scope of the limits of error.

### Example 7

Performance of a Continuous Asymmetrical PCR Amplification and Hybridization Reaction with Addition of Structure Breaker Oligonucleotides at the Beginning of the PCR Reaction.

PCR Amplification and Hybridization

An asymmetrical Cy3-labeled PCR with a clinical DNA sample as template served as target for the hybridization (KDL31, U. Meyer, Biozentrum Basel, Switzerland). The same forward and reverse primers as in Example 6 were used. Regarding the sequence, the same structure breakers were likewise employed. However, these had an amino modification (NH₂ modification) at their 3′-end.

The PCR reaction setup had the following composition and final concentration:

| | |
|---|---|
| 70nM | forward primer: cyp2D6_5/6f |
| | (5′-GGACTCTGTACCTCCTATCCACGTCA-3′) |
| 130 nM | competitor: cyp2D6_5/6f_3NH2 |
| | (5′-GGACTCTGTACCTCCTATCCACGTCA-NH₂-3′) |
| 200 nM | reverse primer: cyp2D6_5/6r_5Cy3 |
| | (5′-Cy3-CCCTCGGCCCCTGCACTGTTTCCCAGA-3′) |
| 20 nM | structure breaker 2938BL5 |
| | (5′GGAACCCTGAGAGCAGCTTC-NH₂-3′) |
| 20 nM | structure breaker 2938BL3 |
| | (5′GGCTGACCTGTTCTCTGCCG-NH₂-3′) |
| 200 μM | dNTPs |
| 1 x buffer | 10 x cDNA reaction buffer (Clontech, |
| | Palo Alto, USA), 70 mMK-acetate |
| 15 U | MasterTaq DNA polymerase (Eppendorf AG, |
| | Hamburg, Germany), |
| 1 ng/μl | template DNA (human genomic DNA KDL 31 |
| | (Urs Meyer, Biozentrum Basel, |
| | Switzerland) |

The volume of the PCR was 50 μl. A parallel setup had the identical composition regarding all components except the structure breaker oligonucleotides. The latter were not added.

Both setups were provided each with an array of the same layout as in Example 6 and were incubated according to the following temperature protocol:

| | | |
|---|---|---|
| Initial denaturation: | 10 min | 95° C. |
| 30 cycles: | 30 s | 95° C. |
| | 50 s | 62° C. |
| | 90 s | 72° C. |
| Terminal elongation: | 7 min | 72° C. |

Hybridization was performed as follows:

| | | |
|---|---|---|
| Denaturation: | 5 min | 95° C. |
| Hybridization: | 60 min | 40° C. |

Subsequently to the hybridization, while being shaken, the chips were washed for 10 min at 30° C. in 2×SSC, 0.2% SDS, at 30° C. in 2×SSC and at 20° C. in 0.2×SSC (Sambrook et al., vide supra) and dried by means of compressed air.

Detection of the Hybridization and Evaluation

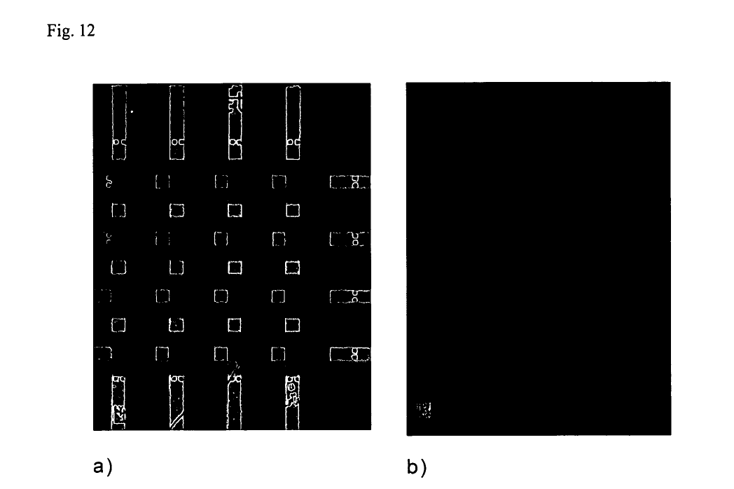

The hybridized and washed chips were read out in a slide scanner (Scanarray4000, GSI Lumonics). Both arrays were recorded with identical scanner settings (Laser-Power 70 and Photomultiplier 80). FIG. 12 (FIG. 12) shows the corresponding scanner pictures. FIG. 12a shows the fluorescence signals after hybridization of a PCR with addition of structure breakers; FIG. 12b, however, shows the reaction without addition of the corresponding structure breakers.

Results

It is becoming clear that the addition of the structure breakers at the beginning of the PCR reaction had no negative influence on the result of the reaction. Rather, a more sensitive detection of the hybridization signals was possible with the addition of the structure breaker oligonucleotides.

### Example 8

Comparison of Asymmetrical and Symmetrical Amplification in a Continuous PCR Amplification and Hybridization Reaction.

A target sequence from Corynebacterium glutamicum was examined for the presence of insertion or deletions against a probe array and by means of hybridization.

Array

A DNA array was produced by means of site-specific in situ synthesis of oligonucleotides. Altogether, the array contained 64 array elements of a size of 256×256 μm. Each array element contained one of the following probes.

| | |
|---|---|
| Insertion: | 5′GTTTCCCAGGGCTTATCCC-3′ |
| Deletion: | 5′-GTTTCCCAGCTTATCCC-3′ |
| Match: | 5′-GTTTCCCAGGCTTATCCC-3′ |

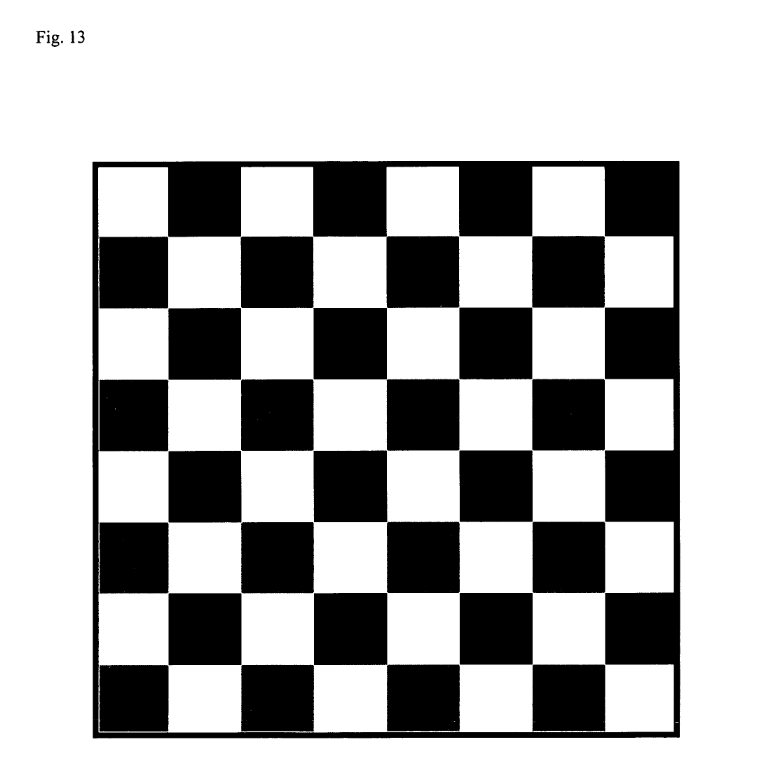

The assignment of the individual probes to the array elements is represented in FIG. 13. Array elements occupied by the match probe are depicted in white, probe elements occupied by the deletion probe are depicted in black, and probe elements occupied by the insertion probe are depicted in gray.

Continuous PCR and Hybridization Reaction

The forward primer 16sfD15′Cy3 had the following sequence and was labeled with the fluorescent colorant Cy3 (Amersham-Pharmacia, Freiburg, Germany) at its 5′-end:

5′-AGAGTTTGATCCTGGCTCAG-3′

The reverse primer 16sRa and the competitor 16sRa3′NH₂ had the following sequence:

5′-TACCGTCACCATAAGGCTTCGTCCCTA-3′

At its 3′-end, the competitor had an amino modification (NH₂), which was integrated into the molecule during the chemical synthesis of the oligonucleotide. 5 symmetrical and 5 asymmetrical reaction setups of the following composition were prepared in each case. The concentrations of the stock solutions are given in parentheses in each case.

Asymmetrical Reactions:

| | |
|---|---|
| 0.5 μl | reverse primer 16sRa (10 μM) |
| 1 μl | c 16SRa3′NH₂ (10 μM) |
| 1.5 μl | 16SfD15′Cy3 (10 μM) |
| 0.75 μl | dNTP mixture (20 mM) |
| 0.75 μl | chromosomal DNA Corynebacterium glutamicum (10 ng/μl) |
| 0.75 μl | 10 × Clontech cDNA buffer |
| 54.5 μl | PCR grade water |
| 3 μl | Taq polymerase (Eppendorf, Hamburg, Germany) (5 U/μl) |
| 5 μl | 1M potassium acetate |

Symmetrical Reactions:

| | | |
|---|---|---|
| 1.5 | μl | reverse primer 16SRa (10 μM) |
| 1.5 | μl | 16SfD15′Cy3 (10 μM) |
| 0.75 | μl | dNTP mixture (20 mM) |
| 0.75 | μl | chromosomal DNA Corynebacterium glutamicum (10 ng/μl) |
| 0.75 | μl | 10 × Clontech cDNA buffer |
| 54.5 | μl | PCR grade water |
| 3 | μl | Taq polymerase (Eppendorf, Hamburg, Germany) (5 U/μl) |
| 5 | μl | 1M potassium acetate |

To the reaction setups in 0.2 ml PCR tubes one array was added in each case. The reaction mixtures including the array were subjected to the following temperature protocol for the PCR amplification and hybridization:

PCR:

| | | |
|---|---|---|
| Initial denaturation | 2 min | 95° C. |
| 10-30 PCR cycles | 30 sec | 95° C. |
| | 30 sec | 62° C. |
| Extension | 30 sec | 72° C. |

The PCR amplification was terminated for a symmetrical and an asymmetrical reaction setup in each case after 10, 15, 20, 25, and 30 cycles, respectively. Hybridization was performed immediately afterwards.

Hybridization:

| | | |
|---|---|---|
| Denaturation | 30 sec | 95° C. |
| Hybridization | 60 min | 42° C. |

Evaluation of the Hybridization

After completion of the hybridization, the arrays were once washed in 0.2×SSC for a short time at room temperature. After removing the liquid, the arrays were dried and read out in a fluorescence scanner (Scanarray4000, GSI Lumonics). The settings of laser and PMT varied depending on the intensity of the hybridization signals.

Results

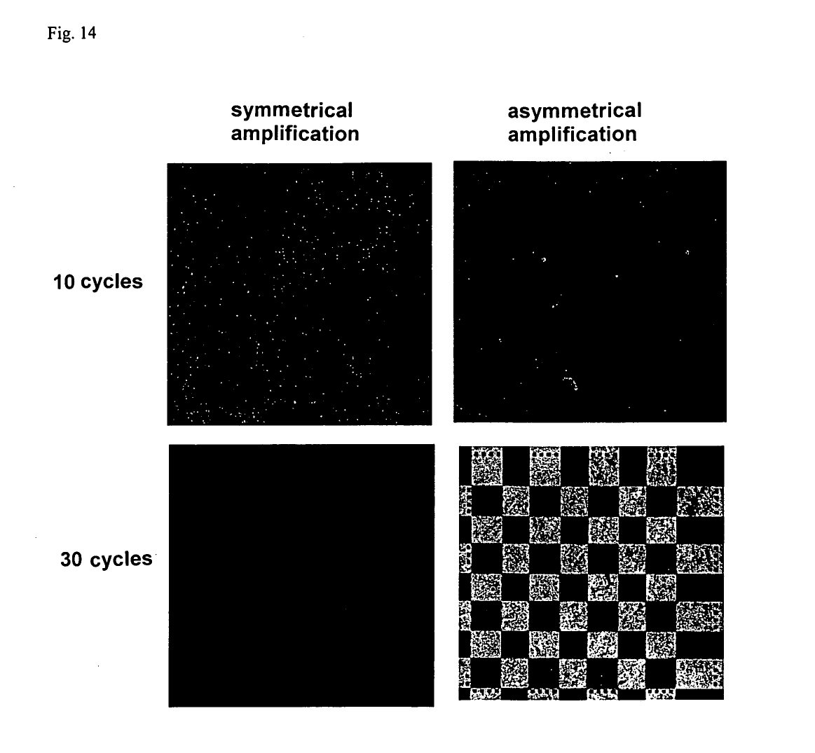

It showed that the asymmetrical reaction in comparison with the symmetrical reaction always led to a stronger hybridization signal, no matter over how many cycles the amplification was performed. As an example, the hybridization signals of the reaction terminated after 10 and 30 cycles, respectively, can be seen in FIG. 14.

### Example 9

Addition of a Molecule Simultaneously Acting as Competitor and Secondary Structure Breaker at the Beginning of a Continuous PCR and Hybridization Reaction

This example shows that the addition of a molecule simultaneously acting as competitor and secondary structure breaker at the beginning of a continuous PCR and hybridization reaction leads to higher hybridization signals.

Secondary structure breakers are oligonucleotides binding in the proximity of the actual hybridization probe in the hybridization of a double-stranded nucleic acid. Thereby, the binding capability of the hybridization probe is improved. Competitors are molecules, which inhibit the formation of the opposite strand, during the PCR amplification by means of binding to one of the complementary strands. The following example shows that one type of molecule can exhibit the functionality of both the competitor and of the structure breaker.

Production of the Array

On a glass wafer by Schott, which was epoxidized in a standard process at Clondiag, 397 DNA probe arrays each having 15 oligonucleotides of different sequence and length were synthetically produced by means of micro wet painting (Clondiag). All oligos were complementary to a partial sequence of the exon 5/6 of the human cyp2D6 gene and differed only due to insertion of several different mutations in the mid-region of the probe. Subsequently to the synthesis, the wafer was sawed up into chips the size of 3.4×3.4 mm, wherein each of these chips contained a probe array with all 15 oligonucleotides in different redundancies. Altogether, each array consisted of 256 spots. These were arranged in 16 identical fields of 16 spots. The following sequences were set up in the individual spots.

| | |
|---|---|
| C2938T | AATGATGAGAACCTGTGCATAGTGGTG |
| C2938TC | AATGATGAGAACCTGTCGCATAGTGGTG |
| C2938GT | AATGATGAGAACCTGGTGCATAGTGGTG |
| C2938GTC | AATGATGAGAACCTGGTCGCATAGTGGTG |
| C2938G | AATGATGAGAACCTGGGCATAGTGGTG |
| C2938GC | AATGATGAGAACCTGGCGGATAGTGGTG |
| C2938d | AATGATGAGAACGTGGCATAGTGGTG |
| C2938CT | AATGATGAGAACCTGCTGCATAGTGGTG |
| C2938CTC | AATGATGAGAACCTGCTCGCATAGTGGTG |
| C2938 | AATGATGAGAAGCTGCGCATAGTGGTG |
| C2938CC | AATGATGAGAACCTGCCGCATAGTGGTG |
| C2938AT | AATGATGAGAACCTGATGCATAGTGGTG |
| C2938ATC | AATGATGAGAACCTGATCGCATAGTGGTG |
| C2938A | AATGATGAGAACCTGAGCATAGTGGTG |
| C2938AC | AATGATGAGAACCTGACGCATAGTGGTG |

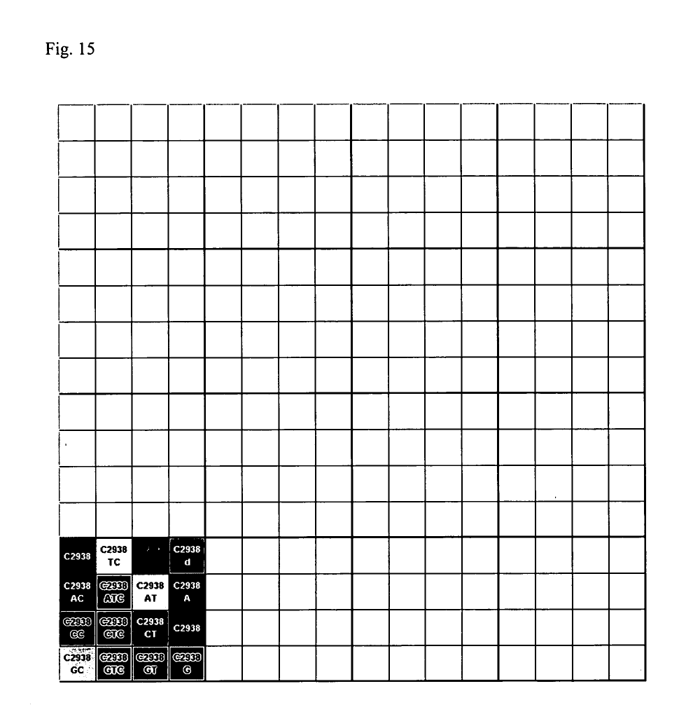

The setup of the array is shown in FIG. 15.

PCR and Hybridization Reaction

A fragment of the human cyp2D6 gene was amplified in the presence of a DNA array and subsequently directly hybridized with the array. The reaction setups (50 μl each) had the following composition:

All reactions contained
1× Advantage PCR reaction buffer (Clontech, Palo Alto, USA)
2× Advantage cDNA polymerase mixture (BD Bioscience, Palo Alto, USA)
chromosomal DNA KDL 31 (0.1 ng/μl final concentration)
200 μM dNTPs (each 200 μM dATP, dTTP, dCTP, dGTP)
additionally 35 mM (final concentration) potassium acetate (Fluka)
Primer cyp2D6_{—}5/6r_{—}5Cy3, final concentration 200 nM

In addition, the reactions contained the following components:

Setup 1:

Primer cyp2D6_{—}5/6f, final concentration 66 nM
Competitor cyp2D6_{—}5/6f 3NH2, final concentration 132 nM
   Setup 2:
Primer cyp2D6_{—}5/6f, final concentration 66 nM
Competitor cyp2D6_{—}5/6f_{—}3NH2, final concentration 132 nM
Structure breaker/competitor C2938T BL5_{—}3′NH2, final concentration 2 μM
   Setup 3:
Primer cyp2D6_{—}5/6f, final concentration 200 nM
Structure breaker/competitor C2938T BL5_{—}3′NH2, final concentration 2 μM
   Setup 4:
Primer cyp2D6_{—}5/6f, final concentration 200 nM
   Setup 5:
Primer cyp2D6_{—}5/6f, final concentration 66 nM
Competitor cyp2D6_{—}5/6f 3NH2, final concentration 132 nM
Structure breaker/competitor C2938T BL5_{—}3′NH2, final concentration 200 nM

The oligonucleotides had the following sequences:

| | |
|---|---|
| Primer cyp2D6_5/6r_5Cy3, | sequence 5′-Cy3-CCCTCGGCGCCTGCACTGTTTCCCAGA-3′, |
| Primer cyp2D6_5/6f, | sequence 5′-GGACTCTGTACCTCCTATCCACGTCA-3′ |
| Competitor cyp2D6_5/6f_3NH2. | sequence 5′-GGACTGTGTACCTCCTATCCACGTCA-NH₂-3′ |
| Structure breaker/competitor C2938T BL5_3′NH2, | sequence 5′-GGAACCCTGAGAGCAGCTTC-NH₂-3′ |

The human chromosomal DNA sample KDL 31 was provided by Prof. U. Meyer, Biozentrum Basel, Switzerland.

PCR Amplification:

The reaction setups (in 0.2 ml PCR tubes) were provided with one array each and subjected to the following temperature regime:
Initial denaturation (2 min, 95° C.)
30 cycles (30 s, 95° C.; 30 s, 62° C.; 150 s, 72° C.)
   Hybridization:

The hybridization was performed immediately after the amplification. To this end, the reaction mixture was incubated together with the array for 3 min at 95° C. and subsequently for 60 min at 42° C.

Subsequently to the hybridization, the arrays were withdrawn from the reaction mixture, briefly rinsed in 500 μl 0.2×SSC (Sambrook et al, 1989) and subsequently dried in a vacuum.

Gel Analysis:

Subsequently to the hybridization, in each case 5 μl of the reaction mixture were withdrawn and electrophoretically separated on a 2% agarose gel (Sambrook et al, 1989). The gel was stained with ethidium bromide and photographed in a gel documentation system (Geldoc 2000, BioRad, München, Germany).

Detection of the Hybridization and Evaluation

The hybridized and washed chips were read out in a device, which allows the reading out of smaller chips in a slide scanner, in a slide scanner (Scanarray4000, GSI Lumonics). 85% laser and 80% PMT power were employed.

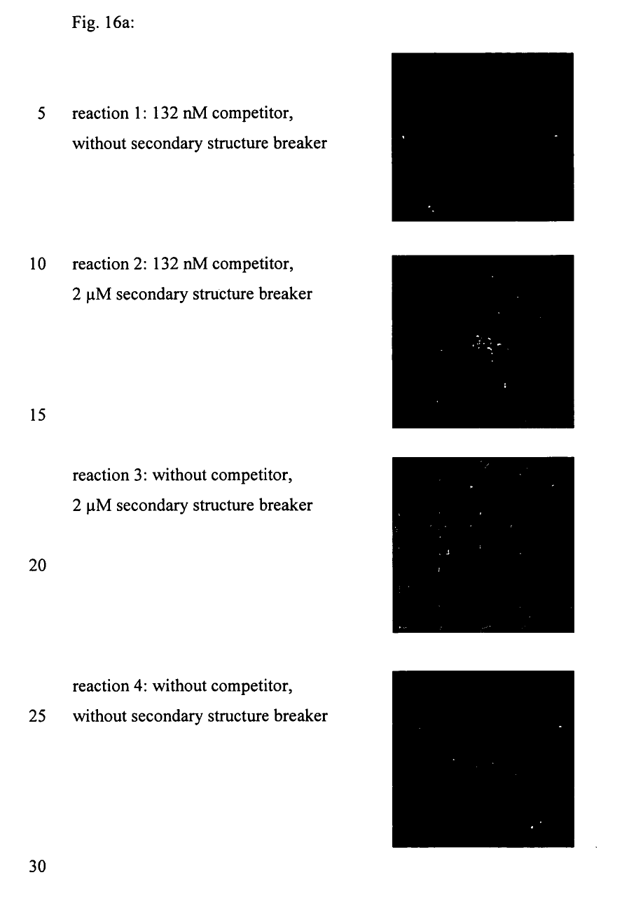

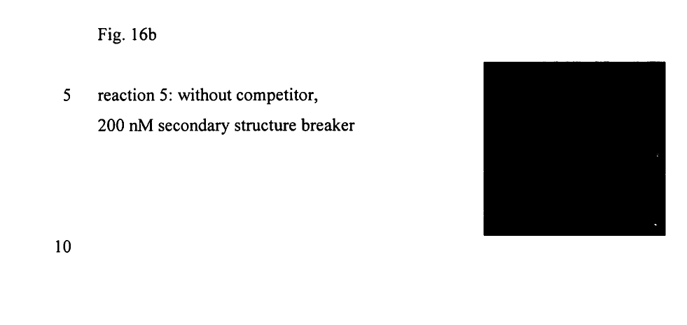

FIG. 16 shows the hybridization results.

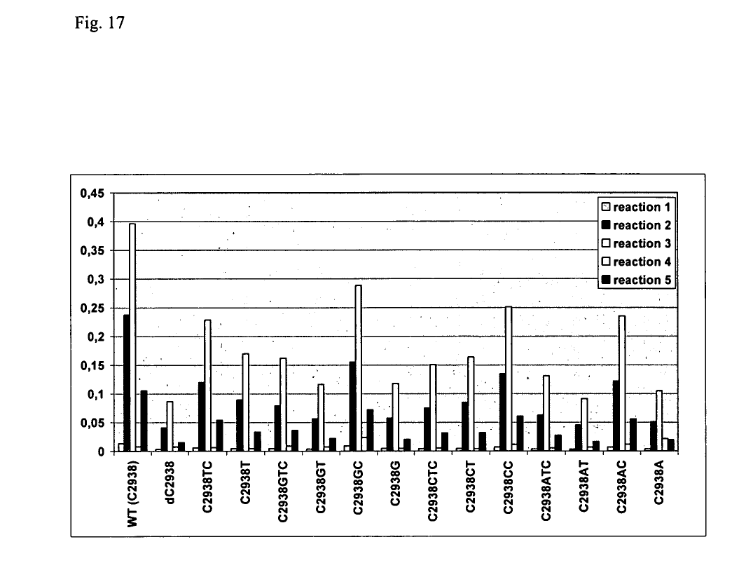

The recorded pictures were evaluated by means of the picture evaluation software Iconoclust® (Clondiag Chip Technologies). The results are depicted in FIG. 17.

The example shows that both in symmetrical and in asymmetrical amplification without addition of structure breakers in a continuous PCR and hybridization method only very weak hybridization signals are obtained, which cannot be discriminated from the background signal. The addition of the secondary structure breaker leads to a strong increase of the hybridization signal. Gel analysis shows that the increase of the hybridization signals does not correlate with the amount of PCR product, which emerged in the reaction. Apparently, the addition of the secondary structure breaker inhibits the product formation in the PCR, as it is to be expected for a competitor, and enhances the hybridization capability of the emerged products, as it is to be expected for a secondary structure breaker.

For the performance of the corresponding reactions see also Sambrook, J., Fritsch, E. F., Maniatis, T. (1989): Molecular Cloning: a laboratory manual, 2^{nd} Edition, Cold Spring Harbor Laboratory Press.

### Example 10

Identification of Fungus Species by Means of Asymmetrical Amplification of a Region of the Ribosomal DNA and Hybridization Against a Microarray.

The detection of the hybridization is performed by means of an enzymatic method, wherein TMB is converted by horseradish peroxidase.

PCR Reactions:

A PCR setup of the following composition is prepared:

| | | |
|---|---|---|
| 10 μM primer IS3: | 0.66 | μl |
| 10 μM primer IS4_5′Bio | 2.0 | μl |
| 10 μM competitor IS3_3′NH2 | 1.33 | μl |
| 20 mM dNTP mixture (each 20 mM dATP, dCTP, | 1.0 | μl |
| dGTP und dTTP) | | |
| chromosomal DNA Aspergillus niger 5331 (10 ng/μl) | 2.0 | μl |
| 10 × Advantage cDNA PCR reaction buffer | 10.0 | μl |
| (Clontech, Palo Alto, U.S.A.) | | |
| PCR grade water | 65 | μl |
| 25 mM Mg-acetate (Eppendorf, Hamburg, Germany) | 6.0 | μl |
| 5 U/μl Taq polymerase (Eppendorf, Hamburg, Germany) | 6.0 | μl |
| 1 M potassium acetate | 6.0 | μl |

The primer and the competitor had the following sequences:

| | |
|---|---|
| IS3: | 5′-GCATCGATGAAGAACGCAGC-3′ |
| IS4_5′Bio: | 5′-Biotin- TCCTCCGCTTATTGATATGC-3′ |
| IS3_3′NH2: | 5′-GCATCGATGAAGAACGCAGC-amino link-3′ |

The reaction setup was filled in two 0.2 μl reaction containers and incubated in a thermocycler (Mastercycler Gradient, Eppendorf, Hamburg, Germany) according to the following temperature regime:

1 cycle:

| | |
|---|---|
| 95° C. | 300 sec |
| 56° C. | 30 sec |
| 72° C. | 60 sec |

30 cycles:

| | |
|---|---|
| 95° C. | 30 sec |
| 56° C. | 30 sec |
| 72° C. | 60 sec |

The reaction mixtures were combined and hybridized against an array tube by Clondiag.

Array Tube:

An array tube (Clondiag Chip Technologies, Jena, Germany) equipped with the following probes in 3-fold redundancy was used:

| | |
|---|---|
| A_Pen_Gen_24 | 5′-CGGTCCTCGAGCGTATGGGGCTTT-amino |
| | link-3′ |
| A.ter_26C | 5′-CCGACGCATTTTTTGTGCAACTTGTT-amino |
| | link-3′ |
| A.ter_26A2 | 5′-CCGAACGCATTTATTTGCAACTTGTT-amino |
| | link-3′ |
| A.ter_25B | 5′-CCGACGCATTTGTTTGCAACTTGTT-amino |
| | link-3′ |
| A.ter_25A1 | 5′-CCGACGCATTTATTTGCAACTTGTT-amino |
| | link-3′ |
| A.nig_24B | 5′-GCCGACGTTTTCCAACCATTCTTT-amino |
| | link-3′ |
| A.nig_23A | 5′-GCCGACGTTATCCAACCATTTTT-amino |
| | link-3′ |
| A.fum_24C | 5′-CAGCCGACACCCAACTTTATTTTT-amino |
| | link-3′ |
| A.fum_24B | 5′-CCAGCCGACACCCAACTTTATTTT-amino |
| | link-3′ |
| A.fum_24A | 5′-GCCAGCCGACACCCAACTTTATTT-amino |
| | link-3′ |
| A.fla_25C | 5′-CCGAACGCAAATCAATCTTTTTCCA-amino |
| | link-3′ |
| A.fla_25A | 5′-CTTGCCGAACGCAAATCAATCTTTT-amino |
| | link-3′ |
| A.fla_24B | 5′-GCCGAACGCAAATCAATCTTTTTC-amino |
| | link-3′ |

Hybridization and Processing of the Array Tube:

The array tube was washed twice with 200 μl deionized water (and) subsequently incubated for 15 min with 2% milk powder in 6×SSPE 0.005% Triton at 30° C. The combined PCR reactions were denatured for 5 min at 95° C. The array tube was emptied, pre-heated to 40° C. and subsequently filled with the PCR. The array tube was incubated for one hour at 40° C. and 550 rpm in a thermo shaker (Eppendorf, Hamburg, Germany). Subsequently, the array tube was consecutively washed for 5 min at 20° C. and 550 rpm in each case with the following solutions:
2×SSC/0.01% Triton,
2×SSC,
0.2×SSC.

Subsequently, the array tube was incubated for 30 min at 30° C. and 550 rpm with a 1:10000-dilution of poly-HRP-streptavidin (Pierce, Rockford, USA) in 6×SSPE, 0.005% Triton.

Subsequently, three steps were performed, each for 5 min at 20° C. and 550 rpm with:
2×SSC/0.01% Triton,
2×SSC,
0.2×SSC
   Detection and Evaluation of the Hybridization Signals:

The array tube was emptied and subsequently filled with 100 μl True Blue TMB peroxidase substrate (Medac, Wedel, Germany). The array tube was immediately read out in an AT-reader (Clondiag Chip Technologies, Jena, Germany). Herein, the temperature was 25° C. A series of pictures of 70 pictures at intervals of 10 s was taken.

The picture series was subsequently evaluated by means of the software Iconoclust (Clondiag Chip Technologies, Jena, Germany). Herein, automatic spot recognition was employed. The signal of each spot was corrected against the local background.

Signals were only observed at the probes specific for Aspergillus niger, A.nig_{—}24B and A.nig_{—}23A, and at the probe with the array tube specificity A_Pen_Gen_{—}24. The signals of the remaining probes having specificity for other array tube strains were within the range of the background signal.

### Example 11

The reaction identical to Example 10 was performed in a closed reaction chamber, the Clondiag assay processor (AP) (Clondiag Chip Technologies GmbH, Jena Germany).

The reaction setup and the array layout corresponded to the preceding example.

Processing of the Assay Processor:

The assay processor was filled with the PCR reaction solution by means of the AP-filling station (Clondiag Chip Technologies GmbH, Jena, Germany). Subsequently, a PCR reaction according to the same parameters as in the preceding example was performed in the AP. A denaturation step for 5 min at 95° C. and the subsequent hybridization for 1 hour at 40° C. followed. The assay processor was first rinsed with 500 μl 0.2×SSC and then with 500 μl of a blocking solution (5% fetal calf serum in 6×SSPE, 0.005% Triton (fluctuation rate 450 μl/minute in each case, temperature 20° C.) and at the end of the rinsing the assay processor was incubated for 10 min with the blocking solution. Subsequently, the blocking solution was removed and the assay processor was rinsed with 250 μl of a 1:100 dilution of a HRP-streptavidin complex (Sigma, Deisenhofen, Germany) in 6×SSPE, 0.005% Triton X-100 (fluctuation rate 450 μl/min) and incubated for 30 min at 20° C. with this solution.

The assay processor was rinsed with 1 ml 2×SSC, 0.01% Triton and subsequently with 500 μl 2×SSC (fluctuation rate 450 μl/minute in each case, temperature 20° C.). Subsequently, the reaction chamber was emptied and rinsed with 250 μl True Blue TMB peroxidase substrate (Medac, Wedel, Germany) and subsequently incubated with this substrate.

The detection of the hybridization signals (TMB-precipitation) was performed in incident light (red light LED) with a CCD camera. A picture series of 70 pictures at intervals of 10 s was recorded.

The evaluation of the hybridization results and the results correspond to the preceding example.

### Example 12

Performance of an Asymmetrical RT-PCR and Hybridization in the Clondiag Assay Processor in a Continuous Process

Assay Processor:

An assay processor (Clondiag Chip Technologies) with an array containing the following probes in 4-fold redundancy (depiction of the probes in 5′-3′-direction, all probes had a C7 amino link at their 3′end) was used:

| | |
|---|---|
| Y_bla-47-24 | TTCGGACTCTTTGATGATTCATAA |
| Y_bla-45-23 | TCTTCGGACTCTTTGATGATTCA |
| SalR3b_22 | GGAAGGTGTTGTGGTTAATACC |
| SalR3a_22 | GGAAGGTGTTGTGGTTAATAAC |
| PA-39-20 | CAAATGTTGGGTGAACGGCT |
| PA-35-22 | AAAGCAAATGTTGGGTGAACGG |
| Ms2_2824 | TTCTCGATGGTCCATACCTTAGAT |
| Ms2_2724 | TAGATGCGTTAGCATTAATCAGGC |
| Ms2_2624 | TTAGATGCGTTAGCATTAATCAGG |
| Ms2_2525 | CTTAGATGCGTTAGCATTAATCAGG |
| Ms2_1423 | ATGGTCCATACCTTAGATGCGTT |
| GneR1_22 | AGGCCTTCGGGTTGTAAAGTAC |
| EcoR3_22 | GGAAGGGAGTAAAGTTAATACC |
| BsuR3b_22 | GAACAAGTGCCGTTCGAATAGG |
| BsuR3a_22 | GAACAAGTACCGTTCGAATAGG |
| BsuR1_22 | AGGTTTTCGGATCGTAAAGCTC |
| bla-3-20 | AACGATCGGAGGACCGAAGG |
| bla-1-21 | ACAACGATCGGAGGACGGAAG |
| BanR3_22 | GAACAAGTGCTAGTTGAATAAG |
| BanR1_22 | AGGCTTTCGGGTCGTAAAACTC |

RT-PCR:

An RT-PCR with the following composition was prepared:

| | |
|---|---|
| 10 μM primer MS2_r_5′Cy3 (5′-cyanine3_TCCGGTTGAGGGCTCTATCT-3′) | 1,3 μl |
| 10 μM primer MS2_f (5′-CAACTGGCGCGTACGTAAA-3′) | 0,44 μl |
| 10 μM competitor MS2_f_3′NH2 | |
| (5′-CAACTGGCGCGTACGTAAA-amino link-3′) | 0,86 μl |
| 20 mM dNTP mixture (each 20 mM dATP, dCTP, dGTP, dTTP) | 0,65 μl |
| 100 mM DTT | 3.25 μl |
| 25 mM MgCl₂ | 6.5 μl |
| PCR grade water | 35 μl |
| 1 M potassium acetate | 3,9 μl |
| 5 x titan one tube PCR buffer (Roche, Mannheim, Germany) | 13 μl |
| Titan one tube RT-PCR enzyme mixture (Roche, Mannheim, Germany) | 1,3 μl |
| Template: MS2-RNA (10 ng4/μl) | 1 μl |

The assay processor was filled with the RT-PCR reaction mixture by means of a filling station. Subsequently, an RT-PCR reaction was performed in the AP in the following manner:

RT Reaction:

10 min at 50° C.
   PCR Amplification:
   1 Cycle:
5 min 95° C.
30 sec 60° C.
40 sec 72° C.
   39 Cycles:
30 sec 95° C.
30 sec 60° C.
40 sec 72° C.
   Hybridization:

The reaction mixture was denatured in the AP for 3 min at 95° C. and subsequently hybridized for 1 h at 40° C. A rinsing step with 500 μl 2×SSC at 20° C. and a fluctuation speed of 450 μl/min followed. The assay processor remained filled with 2×SSC after completion of rinsing.

Reading Out of the Hybridization Signals:

The detection of the hybridization signals was performed under a Zeiss fluorescence microscope (Zeiss, Jena, Germany). The stimulation was performed in incident light with a white light source and with a set of filters suitable for Cyanine 3. The signals were recorded with a CCD camera (PCO-Sensicam, Kehlheim, Germany). Exposure time was 10 sec.

The evaluation of the hybridization signals was performed by means of the software Iconoclust (Clondiag Chip Technologies GmbH, Jena, Germany). Hybridization signals over the experimental background were measured at the MS2-specific probes Ms2_{—}2824, Ms2_{—}2724, Ms2_{—}2624, Ms2_{—}2525, Ms2_{—}1423, while no signal was detectable at the other probes.

1. A method for the efficient amplification of at least one template nucleic acid, comprising amplifying a nucleic acid by a polymerase chain reaction (PCR), wherein at least one competitor that inhibits formation of one of the two template strands amplified by the PCR is added at the beginning of the reaction.

2. A method according to claim 1,
wherein the competitor is selected from proteins, peptides, intercalators, aptamers, nucleic acids and nucleic acid analogs.

3. A method according to claim 2,
wherein the competitor is a nucleic acid selected from DNA and/or RNA molecules.

4. A method according to claim 2,
wherein the competitor is a nucleic acid analog selected from PNA, LNA, TNA and/or an nucleic acid analog having a phosphothioate bond.

5. A method according to claim 2,
wherein the competitor is a nucleic acid and/or a nucleic acid analog, wherein the competitor is enzymatically not extendable.

6. A method according to claim 5,
wherein the 3′-end of the competitor nucleic acid and/or the competitor nucleic acid analog is enzymatically not extendable.

7. A method according to claim 6,
wherein the 3′-end of the competitor nucleic acid and/or the competitor nucleic acid analog has no free 3′-OH group.

8. A method according to claim 7,
wherein the competitor nucleic acid and/or of the competitor nucleic acid analog has, at its 3′-end, an amino group, a phosphate group, a biotin moiety, a fluorophor, or a hydrogen atom.

9. A method according to claim 6,
wherein the competitor nucleic acid and/or competitor nucleic acid has at its 3′-end at least one, two, three, four, or five nucleotides, which are not complementary to the corresponding positions in the respective template strand.

10. A method according to claim 1,
wherein the competitor binds to at least one primer used in the PCR.

11. A method according to claim 10,
wherein the competitor is a nucleic acid and/or a nucleic acid analog having a sequence complementary at least in part to one of the primers used in the PCR.

12. A method according to claim 10,
wherein the competitor is covalently linked with the primer.

13. A method according to claim 12,
wherein the covalent link between competitor and primer cannot serve as template strand for polymerases.

14. A method according to claim 10,
wherein the competitor and the primer form a complex and the stability of the complex of the primer with the competitor is lower than the stability of a complex of the primer with its specific primer binding site on the template strand.

15. A method according to claim 14,
wherein the amplification is performed as a two-phase process, wherein in a first phase an annealing temperature is selected at which the primer binds to its specific primer binding site on the template strand, but not to the competitor, and wherein in a second phase an annealing temperature is selected at which binding between primer and competitor also occurs.

16. A method according to claim 1,
wherein the competitor binds one of the template strands.

17. A method according to claim 16,
wherein the competitor competes against one of the primers used for the PCR for binding to one of the template strands.

18. A method according to claim 17,
wherein stability of a complex of the competitor with the specific primer binding site of the primer on the template is lower than stability of a complex of the primer with its specific primer binding site on the template strand.

19. A method according to claim 18,
wherein the amplification is performed as a two-phase process, wherein in the first phase an annealing temperature is selected at which the primer, but not the competitor, binds to the specific binding site of the primer on the template strand, and in a second phase an annealing temperature is selected at which the competitor binds to the specific binding site of the primer on the template occurs.

20. A method according to claim 1,
wherein the competitor simultaneously acts as secondary structure breaker.

21. A method according to claim 1,
wherein a plurality of competitors are added at the beginning of the reaction.

22. A method according to claim 21,
wherein at least one of the competitors exclusively acts as secondary structure breaker.

23. A method according to claim 22,
wherein a first competitor that acts exclusively as a secondary structure breaker, and at least one second competitor that acts inhibitorily with the template strand are added, and wherein a complex of the first competitor with the template strand is less stable than a complex of the second competitor with the template strand.

24. A method according to claim 23,
wherein the amplification is performed as a two-phase process, wherein in a first phase an annealing temperature is selected at which the second competitor, but not the first competitor, binds to a template strand, and wherein in a second phase a temperature is selected at which the first competitor also binds to the template strand.

25. A method according to claim 1,
wherein the competitor is a nucleic acid and/or a nucleic acid analog comprising at least 10 nucleotides.

26. A method according to claim 1,
wherein the competitor is a nucleic acid and/or a nucleic acid analog comprising at least 15 nucleotides.

27. A method according to claim 1,
wherein the competitor is present at the beginning of the reaction in an amount at least equimolar to one of the PCR primers.

28. A method according to claim 27,
wherein a ratio of molar amounts of primer to competitor at the beginning of the reaction is between 0.01 and 0.99.

29. A method according to claim 1,
wherein the template nucleic acid is DNA.

30. A method according to claim 1,
wherein the template nucleic acid is RNA which is transcribed into DNA by means of a reverse transcriptase before the PCR amplification.

31. A method according to claim 1,
wherein the PCR is a multiplex PCR.

32. A method according to claim 1, further comprising detection of the amplified nucleic acid by means of hybridization with a complementary probe.

33. A method according to claim 32,
wherein the amplification of the nucleic acids and their detection are performed by means of hybridization against a probe in a continuous process.

34. A method according to claim 32,
wherein the competitor binds the template strand complementary to the probe.

35. A method according to claim 34,
wherein the competitor binds the template strand complementary to the probe in a region which is not addressed by the probe.

36. A method according to claim 35,
wherein the competitor binds the template strand in immediate proximity of the sequence region which is addressed by the probe.

37. A method according to claim 36,
wherein the competitor is a DNA oligonucleotide which hybridizes with the template strand detected by means of the hybridization in immediate proximity of the sequence region which is addressed by the probe.

38. A method according to claim 35,
wherein the competitor binds the template nucleic acid near the sequence region which is addressed by the probe.

39. A method according to claim 38,
wherein the competitor is a DNA oligonucleotide which hybridizes with the template strand detected by means of the hybridization near the sequence region, which is addressed by the probe.

40. A method according to claim 32,
wherein the at least one competitor comprises a first competitor which competes against one of the PCR primers for binding to the template, and/or at least one second competitor which binds to the target near or in immediate proximity of the sequence detected by the probe, thus acting as a secondary structure breaker.

41. A method according to claim 32,
further comprising addition of at least one competitor exclusively acting as a secondary structure breaker to the reaction mixture only after completion of the PCR.

42. A method according to claim 32,
wherein the competitor comprises a nucleic acid which has a sequence region complementary to a region of the template strand which is not addressed by the hybridization with the probe, and which has at its 3′-end, at least one, two, three, four, or five nucleotides which is/are not complementary to the corresponding positions in the respective template strand.

43. A method according to claim 32,
wherein the PCR and hybridization are performed in a same buffer system.

44. A method according to claim 32,
wherein the PCR and/or hybridization is/are performed in a closed reaction chamber.
